(19) United States
(12) Patent Application Publication
(10) Pub. No.: US 2024/0116880 A1
(43) Pub. Date: Apr. 11, 2024

(54) CERTAIN CHEMICAL ENTITIES, COMPOSITIONS, AND METHODS

(71) Applicant: NeuPharma, Inc, Foster City, CA (US)

(72) Inventor: Xiangping QIAN, Foster City, CA (US)

(21) Appl. No.: 18/255,227

(22) PCT Filed: Dec. 1, 2021
(86) PCT No.: PCT/US21/61375

§ 371 Date: May 31, 2023

Related U.S. Application Data
(60) Provisional application No. 63/120,588, filed on Dec. 2, 2020.

(51) Int. Cl. C07D 241/44

(52) U.S. Cl. CPC C07D 241/44; C07B 2200/13

(57) ABSTRACT

Chemical entities that are novel compounds, their polymorphs, pharmaceutical compositions, and methods of treatment of cancer are described.

### CROSS-REFERENCE TO RELATED APPLICATIONS

This application claims benefit of U.S. Provisional Patent Application No. 63/120,588, filed on Dec. 2, 2020, which is incorporated herein by reference in its entirety.

### FIELD OF THE INVENTION

Described herein are compounds that are Raf kinase inhibitors, methods of making such compounds, pharmaceutical compositions and medicaments comprising such compounds, and methods of using such compounds in the treatment of conditions, diseases, or disorders that would benefit from modulation of Raf kinase activity.

### BACKGROUND OF THE INVENTION

There are at least 400 enzymes identified as protein kinases. These enzymes catalyze the phosphorylation of target protein substrates. The phosphorylation is usually a transfer reaction of a phosphate group from ATP to the protein substrate. The specific structure in the target substrate to which the phosphate is transferred is a tyrosine, serine or threonine residue. Since these amino acid residues are the target structures for the phosphoryl transfer, these protein kinase enzymes are commonly referred to as tyrosine kinases or serine/threonine kinases.

The phosphorylation reactions, and counteracting phosphatase reactions, at the tyrosine, serine and threonine residues are involved in countless cellular processes that underlie responses to diverse intracellular signals (typically mediated through cellular receptors), regulation of cellular functions, and activation or deactivation of cellular processes. A cascade of protein kinases often participate in intracellular signal transduction and are necessary for the realization of these cellular processes. Because of their ubiquity in these processes, the protein kinases can be found as an integral part of the plasma membrane or as cytoplasmic enzymes or localized in the nucleus, often as components of enzyme complexes. In many instances, these protein kinases are an essential element of enzyme and structural protein complexes that determine where and when a cellular process occurs within a cell.

The identification of effective small compounds which specifically inhibit signal transduction and cellular proliferation by modulating the activity of tyrosine and serine/threonine kinases to regulate and modulate abnormal or inappropriate cell proliferation, differentiation, or metabolism is therefore desirable. In particular, the identification of compounds that specifically inhibit the function of a kinase which is essential for processes leading to cancer would be beneficial.

### SUMMARY OF THE INVENTION

In one aspect, the disclosure provides crystalline N-(2,4,5-trifluoro-3-(3-morpholinoquinoxaline-6-carbonyl)phenyl)propane-1-sulfonamide (Compound A):

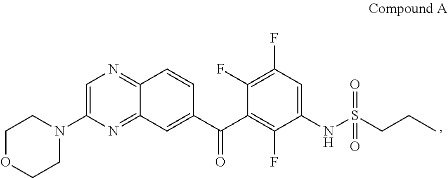 [SMILES: CCCS(=O)(=O)Nc1cc(F)c(F)c(C(=O)c2ccc3ncc(N4CCOCC4)nc3c2)c1F]

or a pharmaceutically acceptable solvate or hydrate thereof.

In some embodiments, the crystalline form is Crystalline Form I. In some embodiments, Crystalline Form I is characterized by:
(a) an X-ray powder diffraction pattern comprising peaks at 8.7±0.2° 2-θ, 21.6±0.2° 2-θ, and 24.4±0.2° 2-θ, as measured by X-ray powder diffraction using an X-ray wavelength of 1.5406 Å;
(b) an X-ray powder diffraction pattern substantially the same as shown in FIG. 1;
(c) a differential scanning calorimetry (DSC) thermogram comprising an endotherm in the range of about 180-190° C.;
(d) a differential scanning calorimetry (DSC) thermogram comprising an endotherm with an onset of about 184° C. and a peak of about 187° C.;
(e) a differential scanning calorimetry (DSC) thermogram substantially the same as shown in FIG. 2;
(f) a Thermogravimetric Analysis (TGA) thermogram substantially the same as shown in FIG. 3;
(g) an unchanged XRPD after storage at 40° C. and 75% relative humidity (RH) for 6 months;
(h) an unchanged XRPD after storage at 25° C. and 60% relative humidity (RH) for 6 months;
or
(i) combinations thereof.

In some embodiments, Crystalline Form I is characterized by an X-ray powder diffraction pattern comprising peaks at 8.7±0.2° 2-θ, 21.6±0.2° 2-θ, and 24.4±0.2° 2-θ, as measured by X-ray powder diffraction using an X-ray wavelength of 1.5406 Å. In some embodiments, the X-ray powder diffraction pattern further comprises at least one peak selected from 17.5±0.2° 2-θ, 14.6±0.2° 2-θ, and 19.3±0.2° 2-θ, as measured by X-ray powder diffraction using an X-ray wavelength of 1.5406 Å. In some embodiments, the X-ray powder diffraction pattern further comprises at least one peak selected from 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises at least five peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises peaks at 8.7±0.1° 2-θ, 21.6±0.1° 2-θ, 24.4±0.1° 2-θ, 17.5±0.1° 2-θ, 14.6±0.1° 2-θ, 19.3±0.1° 2-θ, 23.1±0.1° 2-θ, 16.0±0.1° 2-θ, and 25.7±0.1° 2-θ, as measured by X-ray powder diffraction using an X-ray wavelength of 1.5406 Å. In some embodiments, Crystalline Form I is characterized by an X-ray powder diffraction pattern substantially the same as shown in FIG. 1.

In some embodiments, Crystalline Form I is characterized by a differential scanning calorimetry (DSC) thermogram comprising an endotherm in the range of about 180-190° C. In some embodiments, Crystalline Form I is characterized by a differential scanning calorimetry (DSC) thermogram comprising an endotherm with an onset of about 184° C. and a peak of about 187° C. In some embodiments, Crystalline Form I is characterized by a differential scanning calorimetry (DSC) thermogram substantially the same as shown in FIG. 2.

In some embodiments, Crystalline Form I is characterized by a Thermogravimetric Analysis (TGA) thermogram substantially the same as shown in FIG. 3.

In some embodiments, the crystalline form is Crystalline Form II. In some embodiments, Crystalline Form II is characterized by:
(a) an X-ray powder diffraction pattern comprising peaks at 11.8±0.2° 2-θ, 8.6±0.2° 2-θ, and 15.1±0.2° 2-θ, as measured by X-ray powder diffraction using an X-ray wavelength of 1.5406 Å;
(b) an X-ray powder diffraction pattern substantially the same as shown in FIG. 4;
(c) a differential scanning calorimetry (DSC) thermogram comprising:
   i) an endotherm in the range of about 135-145° C.;
   ii) an exotherm in the range of about 143-153° C.; and
   iii) an endotherm in the range of 155-165° C.;
(d) a differential scanning calorimetry (DSC) thermogram comprising:
   i) an endotherm with an onset of about 138° C. and a peak of about 143° C.;
   ii) an exotherm with an onset of about 146° C. and a peak of about 148° C.; and
   iii) an endotherm with an onset of about 158° C. and a peak of about 160° C.;
(e) a differential scanning calorimetry (DSC) thermogram substantially the same as shown in FIG. 5;
   or
(f) combinations thereof.

In some embodiments, Crystalline Form II is characterized by an X-ray powder diffraction pattern comprising peaks at 11.8±0.2° 2-θ, 8.6±0.2° 2-θ, and 15.1±0.2° 2-θ, as measured by X-ray powder diffraction using an X-ray wavelength of 1.5406 Å. In some embodiments, the X-ray powder diffraction pattern further comprises at least one peak selected from 17.2±0.2° 2-θ, 9.3±0.2° 2-θ, and 23.6±0.2° 2-θ, as measured by X-ray powder diffraction using an X-ray wavelength of 1.5406 Å. In some embodiments, the X-ray powder diffraction pattern further comprises at least one peak selected from 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises at least five peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises peaks at 11.8±0.1° 2-θ, 8.6±0.1° 2-θ, 15.1±0.1° 2-θ, 17.2±0.1° 2-θ, 9.3±0.1° 2-θ, 23.6±0.1° 2-θ, 21.5±0.1° 2-θ, 22.2±0.1° 2-θ, and 14.3±0.1° 2-θ, as measured by X-ray powder diffraction using an X-ray wavelength of 1.5406 Å. In some embodiments, Crystalline Form II is characterized by an X-ray powder diffraction pattern substantially the same as shown in FIG. 4.

In some embodiments, Crystalline Form II is characterized by a differential scanning calorimetry (DSC) thermogram comprising:
i) an endotherm in the range of about 135-145° C.;
ii) an exotherm in the range of about 143-153° C.; and
iii) an endotherm in the range of 155-165° C.

In some embodiments, Crystalline Form II is characterized by a differential scanning calorimetry (DSC) thermogram comprising:
i) an endotherm with an onset of about 138° C. and a peak of about 143° C.;
ii) an exotherm with an onset of about 146° C. and a peak of about 148° C.; and
iii) an endotherm with an onset of about 158° C. and a peak of about 160° C.

In some embodiments, Crystalline Form II is characterized by a differential scanning calorimetry (DSC) thermogram substantially the same as shown in FIG. 5.

In another aspect, the disclosure provides a pharmaceutical composition comprising crystalline N-(2,4,5-trifluoro-3-(3-morpholinoquinoxaline-6-carbonyl)phenyl)propane-1-sulfonamide and at least one pharmaceutically acceptable excipient. In some embodiments, the pharmaceutical composition is formulated for administration to a mammal by oral administration. In some embodiments, the pharmaceutical composition is in the form of a solid form pharmaceutical composition. In some embodiments, the pharmaceutical composition is in the form of a tablet, a pill, or a capsule.

In another aspect, the disclosure provides a packaged pharmaceutical composition comprising a pharmaceutical composition described herein and instructions for using the composition to treat a subject suffering from cancer.

In another aspect, the disclosure provides a method of treating a neoplasm in a subject in need thereof, comprising administering to the subject a therapeutically effective amount of crystalline N-(2,4,5-trifluoro-3-(3-morpholinoquinoxaline-6-carbonyl)phenyl)propane-1-sulfonamide or a pharmaceutical composition described herein. In some embodiments, the neoplasm is a cancer. In some embodiments, the cancer is colon carcinoma, pancreatic cancer, breast cancer, ovarian cancer, prostate cancer, thyroid cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, thyroid cancer, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung cancer, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, Langerhans cell histiocytosis (LCH), Erdheim-Chester disease (ECD), leukemia, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, or heavy chain disease. In some embodiments, the cancer is melanoma. In some embodiments, the melanoma is unresectable or metastatic melanoma. In some embodiments, the cancer is non-small cell lung cancer. In some embodiments, the cancer is colon cancer. In some embodiments, the cancer is thyroid cancer. In some embodiments, the cancer is ovarian cancer. In some embodiments, the neoplasm is a benign tumor. In some embodiments, the tumor is craniopharyngioma.

In another aspect, the disclosure provides a method of preparing Crystalline Form I of N-(2,4,5-trifluoro-3-(3-morpholinoquinoxaline-6-carbonyl)phenyl)propane-1-sulfonamide, wherein the method comprises:
(a) dissolving the N-(2,4,5-trifluoro-3-(3-morpholinoquinoxaline-6-carbonyl)phenyl)propane-1-sulfonamide in a solvent to obtain a solution; and
(b) crystallizing the solution obtained in step (a) to obtain Crystalline Form I of N-(2,4,5-trifluoro-3-(3-morpholinoquinoxaline-6-carbonyl)phenyl)propane-1-sulfonamide.

In some embodiments, the solvent in step (a) comprises ethyl acetate, DCM, ethanol, or isopropanol. In some embodiments, the solvent in step (a) comprises ethyl acetate. In some embodiments, step (a) is performed at a temperature of about 60-90° C. In some embodiments, step (a) is performed at a temperature of about 75-80° C. In some embodiments, step (a) is performed for a time of about 1-3 hours. In some embodiments, step (a) is performed for a time of about 2 hours.

In some embodiments, step (b) comprises cooling the solution obtained in step (a) to room temperature. In some embodiments, step (b) comprises cooling the solution obtained in step (a) to a temperature of about 20-25° C. In some embodiments, the method further comprises filtering the crystallized solution obtained in step (b) to obtain Crystalline Form I. In some embodiments, the method further comprises drying the obtained Crystalline Form I. In some embodiments, the drying is performed under vacuum at a temperature of about 40-50° C.

In another aspect, the disclosure provides a method of preparing Crystalline Form II of N-(2,4,5-trifluoro-3-(3-morpholinoquinoxaline-6-carbonyl)phenyl)propane-1-sulfonamide, wherein the method comprises:
(a) dissolving the N-(2,4,5-trifluoro-3-(3-morpholinoquinoxaline-6-carbonyl)phenyl)propane-1-sulfonamide in a solvent to obtain a first solution;
(b) adding water to the first solution obtained in step (a) to form a mixture;
(c) separating the mixture obtained in step (b) into an organic phase and aqueous phase;
(d) isolating the solids from the organic phase;
(e) dissolving the solids obtained in step (d) in a second solvent to obtain a second solution; and
(f) crystallizing the second solution obtained in step (e) to obtain Crystalline Form II of N-(2,4,5-trifluoro-3-(3-morpholinoquinoxaline-6-carbonyl)phenyl)propane-1-sulfonamide.

In some embodiments, the solvent of step (a) comprises ethyl acetate, DCM, ethanol, or isopropanol. In some embodiments, the amount of water added in step (b) is approximately 1:1 to 1:10 weight ratio of water:solvent added in step (a). In some embodiments, the amount of water added in step (b) is approximately 1:2 to 1:4 weight ratio of water:solvent added in step (a).

In some embodiments, step (d) comprises concentrating the organic phase. In some embodiments, the concentrating is performed under vacuum at a temperature of about 40-50° C. In some embodiments, the second solvent of step (e) comprises ethyl acetate, DCM, ethanol, or isopropanol. In some embodiments, the second solvent of step (e) is ethyl acetate. In some embodiments, step (f) comprises cooling the second solution obtained in step (e) to a temperature of about 10-20° C. In some embodiments, the solution is maintained at a temperature of about 10-20° C. for about 3-5 hours.

In some embodiments, the method further comprises filtering the crystallized solution obtained in step (f) to obtain Crystalline Form II. In some embodiments, the method further comprises drying the obtained Crystalline Form II. In some embodiments, the drying is performed under vacuum at a temperature of about 50-60° C.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### DESCRIPTION OF THE DRAWINGS

The novel features of the invention are set forth with particularity in the appended claims. An understanding of the features and advantages of the present invention may be obtained by reference to the following detailed description that sets forth illustrative embodiments, in which the principles of the invention are utilized, and the accompanying drawings of which:

FIG. 1 shows the X-ray powder diffraction (XRPD) pattern for Crystalline Form I of Compound A.

FIG. 2 shows the differential scanning calorimetry (DSC) thermogram for Crystalline Form I of Compound A.

FIG. 3 shows the thermogravimetric analysis (TGA) thermogram for Crystalline Form I of Compound A.

FIG. 4 shows the X-ray powder diffraction (XRPD) pattern for Crystalline Form II of Compound A.

FIG. 5 shows the differential scanning calorimetry (DSC) thermogram for Crystalline Form II of Compound A.

FIG. 6 shows the X-ray powder diffraction (XRPD) pattern for Crystalline Form I of Compound A stored at 40° C. and 75% relative humidity (RH) for 6 months.

FIG. 7 shows the differential scanning calorimetry (DSC) thermogram for Crystalline Form I of Compound A stored at 40° C. and 75% relative humidity (RH) for 6 months.

FIG. 8 shows the X-ray powder diffraction (XRPD) pattern for Crystalline Form I of Compound A stored at 25° C. and 60% relative humidity (RH) for 6 months.

FIG. 9 shows the differential scanning calorimetry (DSC) thermogram for Crystalline Form I of Compound A stored at 25° C. and 60% relative humidity (RH) for 6 months.

### DETAILED DESCRIPTION OF THE INVENTION

While small molecule inhibitors are often initially evaluated for their activity when dissolved in solution, solid state characteristics such as polymorphism are also important. Polymorphic forms of a drug substance, such as a kinase inhibitor, can have different physical properties, including melting point, apparent solubility, dissolution rate, optical and mechanical properties, vapor pressure, and density. These properties can have a direct effect on the ability to process or manufacture a drug substance and the drug product. Moreover, differences in these properties can and often lead to different pharmacokinetics profiles for different polymorphic forms of a drug. Therefore, polymorphism is often an important factor under regulatory review of the ‘sameness’ of drug products from various manufacturers. For example, polymorphism has been evaluated in many multi-million dollar and even multi-billion dollar drugs, such as warfarin sodium, famotidine, and ranitidine. Polymorphism can affect the quality, safety, and/or efficacy of a drug product, such as a kinase inhibitor. Thus, there still remains a need for polymorphs of kinase inhibitors. The present disclosure addresses this need and provides related advantages as well.

### Compound A

As used herein, Compound A refers to N-(2,4,5-trifluoro-3-(3-morpholinoquinoxaline-6-carbonyl)phenyl)propane-1-sulfonamide, which has the chemical structure shown below:

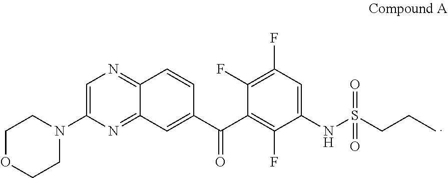 [SMILES: CCCS(=O)(=O)Nc1cc(F)c(F)c(C(=O)c2ccc3ncc(N4CCOCC4)nc3c2)c1F]

Compound A is a potent and selective Raf kinase inhibitor. Raf kinase inhibitors are useful in the treatment of various diseases, conditions, and disorders for which abnormal Raf activity plays a role, such as cancer. In some embodiments, Compound A is a B-Raf inhibitor.

The preparation and uses of Compound A have been previously described (see, WO 2013/032951, U.S. Pat. Nos. 9,295,671, 9,572,808, 10,137,125, and 10,561,652, each of which is incorporated by reference in its entirety).

In some embodiments disclosed herein, Compound A is crystalline.

As used herein, “crystalline form,” “polymorph,” “Form,” and “form” may be used interchangeably herein, and are meant to include all crystalline and amorphous forms of the compound, including, for example, polymorphs, pseudopolymorphs, salts, solvates, hydrates, unsolvated polymorphs (including anhydrates), conformational polymorphs, and amorphous forms, as well as mixtures thereof, unless a particular crystalline or amorphous form is referred to. Compounds of the present disclosure include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. In some embodiments, the crystalline form is a single solid state form, e.g., crystalline Form I.

### I. Crystalline Forms of Compound A

The polymorphs made according to the methods of the invention may be characterized by any methodology according to the art. For example, the polymorphs made according to the methods of the invention may be characterized by X-ray powder diffraction (XRPD), differential scanning calorimetry (DSC), thermogravimetric analysis (TGA), hot-stage microscopy, and/or spectroscopy (e.g., Raman, solid state nuclear magnetic resonance (ssNMR), and infrared (IR)). In some embodiments, crystallinity of a solid form is determined by X-Ray Powder Diffraction (XRPD).

XRPD: Polymorphs according to the invention may be characterized by XRPD. The relative intensities of XRPD peaks can vary, depending upon the particle size, the sample preparation technique, the sample mounting procedure and the particular instrument employed. Moreover, instrument variation and other factors can affect the 2-θ values. Therefore, the XRPD peak assignments can vary, for example by plus or minus about 0.2 degrees.

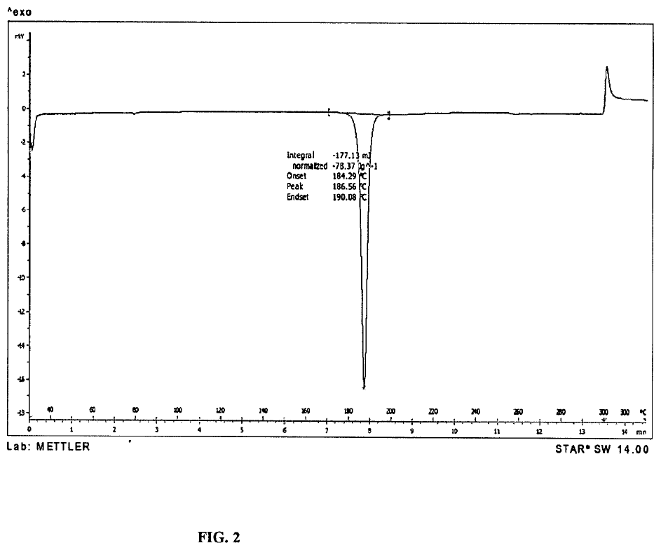

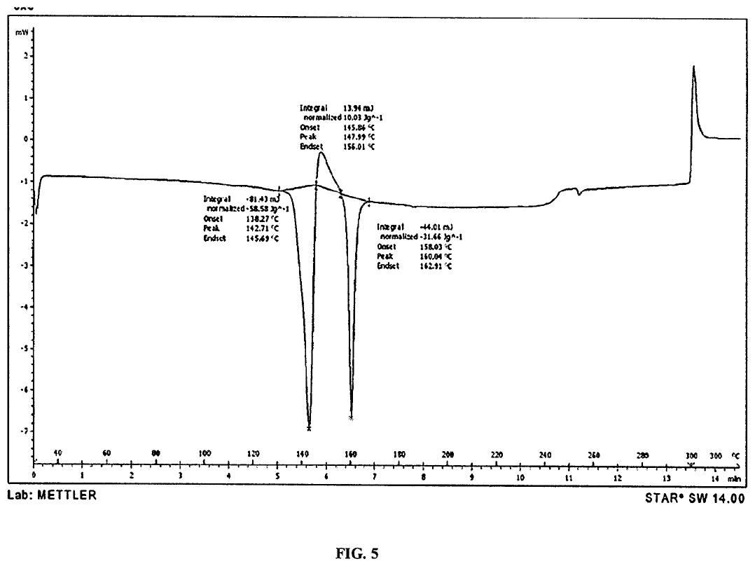

DSC: Polymorphs according to the invention can also be identified by its characteristic DSC thermograms such as shown in FIGS. 2, 5 etc. For DSC, it is known that the temperatures observed will depend upon the rate of temperature change as well as sample preparation technique and the particular instrument employed. Thus, the values reported herein relating to DSC thermograms can vary, for example by plus or minus about 4° C.

TGA: The polymorphic forms of the invention may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the polymorph may be characterized by thermogravimetric analysis.

The polymorph forms of Compound A are useful in the production of medicinal preparations and can be obtained by means of a crystallization process to produce crystalline and semi-crystalline forms or a solidification process to obtain the amorphous form. In various embodiments, the crystallization is carried out by either generating the desired compound (for example Compound A) in a reaction mixture and isolating the desired polymorph from the reaction mixture, or by dissolving raw compound in a solvent, optionally with heat, followed by crystallizing/solidifying the product by cooling (including active cooling) and/or by the addition of an antisolvent for a period of time. The crystallization or solidification may be followed by drying carried out under controlled conditions until the desired water content is reached in the end polymorphic form.

In various embodiments, the various polymorph Forms disclosed herein (e.g., Crystalline Form I and Crystalline Form II of Compound A) are stable at room temperature. In some examples, the various polymorphs can be stored at room temperature for an extended period of time without significant chemical degradation or change in the crystalline form. In some examples, the various polymorphs can be stored at room temperature for a time period of at least about 10 days, 30 days, 60 days, 90 days, 120 days, 150 days, or 180 days. In some examples, the various polymorphs can be stored at room temperature for a time period of more than about 180 days. In some examples, the various polymorphs can be stored at room temperature for a time period of 10-14 days, 10-18 days, 10-22 days, 10-26 days, 10-30 days, 10-40 days, 10-50 days, 10-60 days, 10-90 days, 10-120 days, 10-150 days, 10-180 days, 14-18 days, 14-22 days, 14-26 days, 14-30 days, 14-40 days, 14-50 days, 14-60 days, 14-90 days, 14-120 days, 14-150 days, 14-180 days, 18-22 days, 18-26 days, 18-30 days, 18-40 days, 18-50 days, 18-60 days, 18-90 days, 18-120 days, 18-150 days, 18-180 days, 22-26 days, 22-30 days, 22-40 days, 22-50 days, 22-60 days, 22-90 days, 22-120 days, 22-150 days, 22-180 days, 26-30 days, 26-40 days, 26-50 days, 26-60 days, 26-90 days, 26-120 days, 26-150 days, 26-180 days, 30-40 days, 30-50 days, 30-60 days, 30-90 days, 30-120 days, 30-150 days, 30-180 days, 40-50 days, 40-60 days, 40-90 days, 40-120 days, 40-150 days, 40-180 days, 50-60 days, 50-90 days, 50-120 days, 50-150 days, 50-180 days, 60-90 days, 60-120 days, 60-150 days, 60-180 days, 90-120 days, 90-150 days, or 90-180 days. In some examples, the various polymorphs can be stored at room temperature for a time period of at least 10 days, 14 days, 18 days, 22 days, 26 days, 30 days, 40 days, 50 days, 60 days, 90 days, 120 days, 150 days, or 180 days.

In various embodiments, the various polymorph Forms disclosed herein (e.g., Crystalline Form I and Crystalline Form II of Compound A) are stable at temperatures above the room temperature and/or at high relative humidity (RH). In some examples, the various polymorph Forms disclosed herein (e.g., Crystalline Form I and Crystalline Form II of Compound A) can be stored at about 40° C. at about 75% RH for an extended period of time without significant chemical degradation or change in the crystalline form. In some examples, the various polymorph Forms disclosed herein (e.g., Crystalline Form I and Crystalline Form II of Compound A) can be stored at 40° C. and at about 75% RH for a time period of at least about 10 days, 30 days, 60 days, 90 days, 120 days, 150 days, or 180 days. In some examples, the various polymorph Forms disclosed herein (e.g., Crystalline Form I and Crystalline Form II of Compound A) can be stored at 40° C. and at about 75% RH for a time period of more than about 180 days. In some examples, the various polymorph Forms disclosed herein (e.g., Crystalline Form I and Crystalline Form II of Compound A) can be stored at 40° C. and at about 75% RH for a time period of 10-14 days, 10-18 days, 10-22 days, 10-26 days, 10-30 days, 10-40 days, 10-50 days, 10-60 days, 10-90 days, 10-120 days, 10-150 days, 10-180 days, 14-18 days, 14-22 days, 14-26 days, 14-30 days, 14-40 days, 14-50 days, 14-60 days, 14-90 days, 14-120 days, 14-150 days, 14-180 days, 18-22 days, 18-26 days, 18-30 days, 18-40 days, 18-50 days, 18-60 days, 18-90 days, 18-120 days, 18-150 days, 18-180 days, 22-26 days, 22-30 days, 22-40 days, 22-50 days, 22-60 days, 22-90 days, 22-120 days, 22-150 days, 22-180 days, 26-30 days, 26-40 days, 26-50 days, 26-60 days, 26-90 days, 26-120 days, 26-150 days, 26-180 days, 30-40 days, 30-50 days, 30-60 days, 30-90 days, 30-120 days, 30-150 days, 30-180 days, 40-50 days, 40-60 days, 40-90 days, 40-120 days, 40-150 days, 40-180 days, 50-60 days, 50-90 days, 50-120 days, 50-150 days, 50-180 days, 60-90 days, 60-120 days, 60-150 days, 60-180 days, 90-120 days, 90-150 days, or 90-180 days. In some examples, the various polymorph Forms disclosed herein (e.g., Crystalline Form I and Crystalline Form II of Compound A) can be stored at 40° C. at about 75% RH for a time period of at least 10 days, 14 days, 18 days, 22 days, 26 days, 30 days, 40 days, 50 days, 60 days, 90 days, 120 days, 150 days, or 180 days.

### Crystalline Form I of Compound A

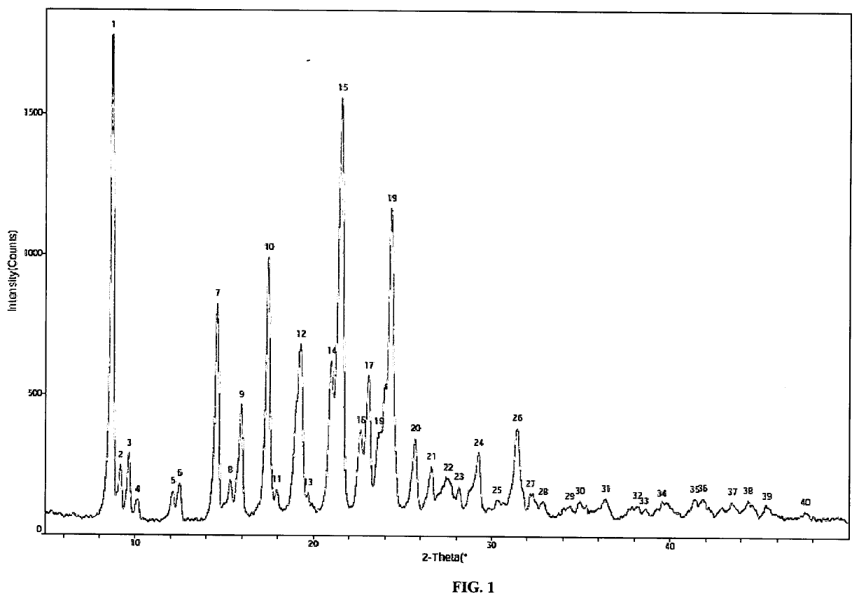

FIG. 1 shows the X-ray powder diffraction (XRPD) pattern for Crystalline Form I of Compound A.

FIG. 2 shows the differential scanning calorimetry (DSC) thermogram for Crystalline Form I of Compound A.

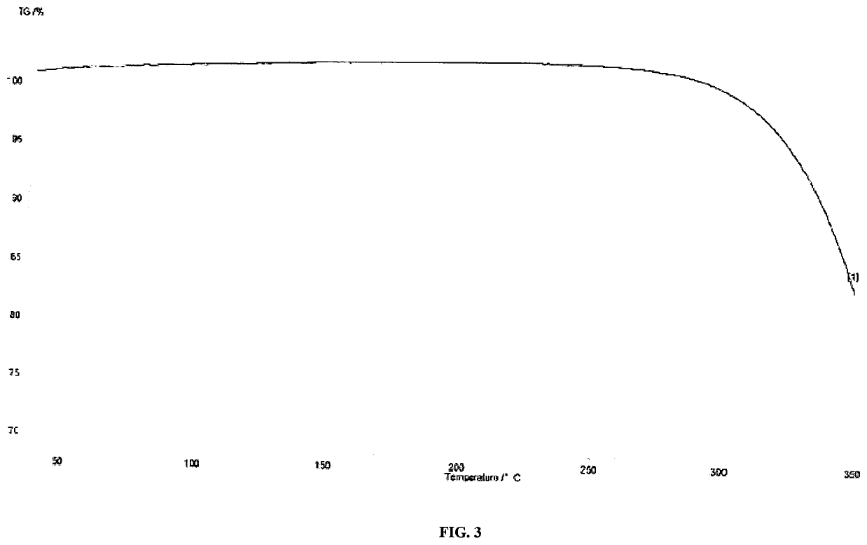

FIG. 3 shows the thermogravimetric analysis (TGA) thermogram for Crystalline Form I of Compound A.

In one aspect, provided herein is Crystalline Form I of N-(2,4,5-trifluoro-3-(3-morpholinoquinoxaline-6-carbonyl)phenyl)propane-1-sulfonamide. Some embodiments provide a composition comprising Crystalline Form I of N-(2,4,5-trifluoro-3-(3-morpholinoquinoxaline-6-carbonyl)phenyl)propane-1-sulfonamide. In some embodiments, Crystalline Form I of N-(2,4,5-trifluoro-3-(3-morpholinoquinoxaline-6-carbonyl)phenyl)propane-1-sulfonamide is characterized as having:
(a) an X-ray powder diffraction pattern comprising peaks at 8.7±0.2° 2-θ, 21.6±0.2° 2-θ, and 24.4±0.2° 2-θ, as measured by X-ray powder diffraction using an X-ray wavelength of 1.5406 Å;
(b) an X-ray powder diffraction pattern substantially the same as shown in FIG. 1;
(c) a differential scanning calorimetry (DSC) thermogram comprising an endotherm in the range of about 180-190° C.;
(d) a differential scanning calorimetry (DSC) thermogram comprising an endotherm with an onset of about 184° C. and a peak of about 187° C.;
(e) a differential scanning calorimetry (DSC) thermogram substantially the same as shown in FIG. 2;
(f) a Thermogravimetric Analysis (TGA) thermogram substantially the same as shown in FIG. 3;
(g) an unchanged XRPD after storage at 40° C. and 75% relative humidity (RH) for 6 months;
(h) an unchanged XRPD after storage at 25° C. and 60% relative humidity (RH) for 6 months; or
(i) combinations thereof.

In some embodiments, Crystalline Form I is characterized by an X-ray powder diffraction pattern substantially the same as shown in FIG. 1.

In some embodiments, Crystalline Form I is characterized by an X-ray powder diffraction pattern comprising peaks at 8.7±0.2° 2-θ, 21.6±0.2° 2-θ, and 24.4±0.2° 2-θ, as measured by X-ray powder diffraction using an X-ray wavelength of 1.5406 Å. In some embodiments, Crystalline Form I is characterized by an X-ray powder diffraction pattern comprising peaks at 8.7±0.1° 2-θ, 21.6±0.1° 2-θ, and 24.4±0.1° 2-θ, as measured by X-ray powder diffraction using an X-ray wavelength of 1.5406 Å. In some embodiments, Crystalline Form I is characterized by an X-ray powder diffraction pattern comprising peaks at about 8.7° 2-θ, about 21.6° 2-θ, and about 24.4° 2-θ, as measured by X-ray powder diffraction using an X-ray wavelength of 1.5406 Å.

In some embodiments, the X-ray powder diffraction pattern further comprises at least one peak selected from 17.5±0.2° 2-θ, 14.6±0.2° 2-θ, and 19.3±0.2° 2-θ, as measured by X-ray powder diffraction using an X-ray wavelength of 1.5406 Å. In some embodiments, the X-ray powder diffraction pattern further comprises at least one peak selected from 17.5±0.1° 2-θ, 14.6±0.1° 2-θ, and 19.3±0.1° 2-θ, as measured by X-ray powder diffraction using an X-ray wavelength of 1.5406 Å. In some embodiments, the X-ray powder diffraction pattern further comprises at least one peak selected from about 17.5° 2-θ, about 14.6° 2-θ, and about 19.3° 2-θ, as measured by X-ray powder diffraction using an X-ray wavelength of 1.5406 Å.

In some embodiments, the X-ray powder diffraction pattern further comprises at least one peak selected from 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern further comprises at least one peak selected from 23.1±0.1° 2-θ, 16.0±0.1° 2-θ, and 25.7±0.1° 2-θ, as measured by X-ray powder diffraction using an X-ray wavelength of 1.5406 Å. In some embodiments, the X-ray powder diffraction pattern further comprises at least one peak selected from about 23.1° 2-θ, about 16.0° 2-θ, and about 25.7° 2-θ, as measured by X-ray powder diffraction using an X-ray wavelength of 1.5406 Å.

In some embodiments, the X-ray powder diffraction pattern comprises at least one peak selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises at least two peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises at least three peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises at least four peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises at least five peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises at least six peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises at least seven peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises at least eight peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises peaks at 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å. In some embodiments, the X-ray powder diffraction pattern comprises peaks at 8.7±0.1° 2-θ, 21.6±0.1° 2-θ, 24.4±0.1° 2-θ, 17.5±0.1° 2-θ, 14.6±0.1° 2-θ, 19.3±0.1° 2-θ, 23.1±0.1° 2-θ, 16.0±0.1° 2-θ, and 25.7±0.1° 2-θ, as measured by X-ray powder diffraction using an X-ray wavelength of 1.5406 Å. In some embodiments, the X-ray powder diffraction pattern comprises peaks at about 8.7° 2-θ, about 21.6 2-θ, about 24.4 2-θ, about 17.5° 2-θ, about 14.6° 2-θ, about 19.3° 2-θ, about 23.1° 2-θ, about 16.0° 2-θ, and about 25.7° 2-θ, as measured by X-ray powder diffraction using an X-ray wavelength of 1.5406 Å.

In some embodiments, Crystalline Form I is characterized by a differential scanning calorimetry (DSC) thermogram substantially the same as shown in FIG. 2. In some embodiments, Crystalline Form I is characterized by a differential scanning calorimetry (DSC) thermogram comprising an endotherm in the range of about 180-190° C. In some embodiments, Crystalline Form I is characterized by a differential scanning calorimetry (DSC) thermogram comprising an endotherm with an onset of about 184° C. and a peak of about 187° C.

In some embodiments, Crystalline Form I is characterized by an endotherm at about 160-180° C., 162-180° C., 164-180° C., 166-180° C., 168-180° C., 170-180° C., 172-180° C., 174-180° C., 160-178° C., 162-178° C., 164-178° C., 166-178° C., 168-178° C., 170-178° C., 172-178° C., 174-178° C., 160-176° C., 162-176° C., 164-176° C., 166-176° C., 168-176° C., 170-176° C., 172-176° C., 174-176° C., 160-174° C., 162-174° C., 164-174° C., 166-174° C., 168-174° C., 170-174° C., 172-174° C., 160-172° C., 162-172° C., 164-172° C., 166-172° C., 168-172° C., 170-172° C., 160-170° C., 162-170° C., 164-170° C., 166-170° C., 168-170° C., 160-168° C., 162-168° C., 164-168° C., 166-168° C., 160-166° C., 162-166° C., 164-166° C., 160-164° C., 162-164° C., 160-162° C. in the DSC thermogram. In various embodiments, Crystalline Form I is characterized by an endotherm at about 180-190° C. in the DSC thermogram, for example about 180° C., 181° C., 181° C., 183° C., 184° C., 185° C., 186° C., 187° C., 188° C., 189° C., or 190° C. In some embodiments, the melting point of Crystalline Form I is about 187° C.

In some embodiments, Crystalline Form I is characterized by a Thermogravimetric Analysis (TGA) thermogram substantially the same as shown in FIG. 3. In various embodiments, Crystalline Form I decomposes above a temperature of about 200° C., about 250° C., about 300° C., about 350° C., about 400° C., about 450° C., about 500° C., about 550° C. or above 600° C. In some examples, Crystalline Form I decomposes above a temperature of about 250° C.

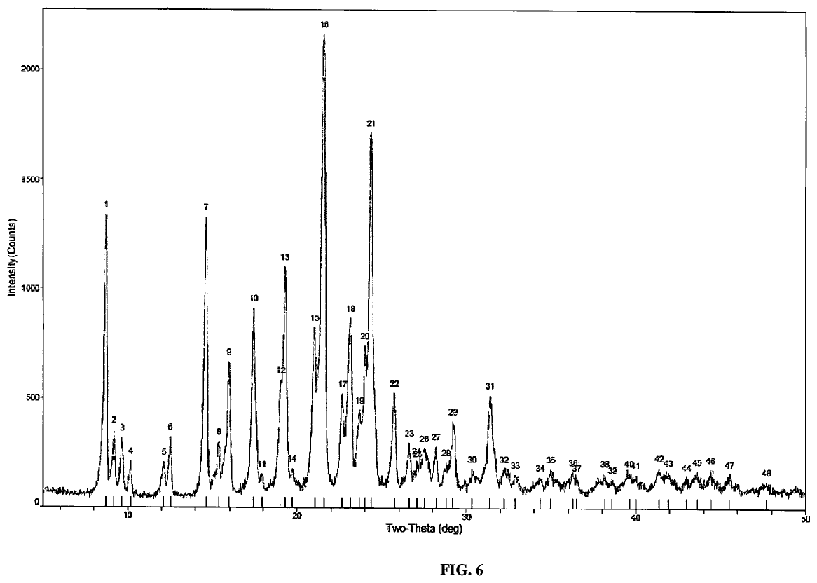

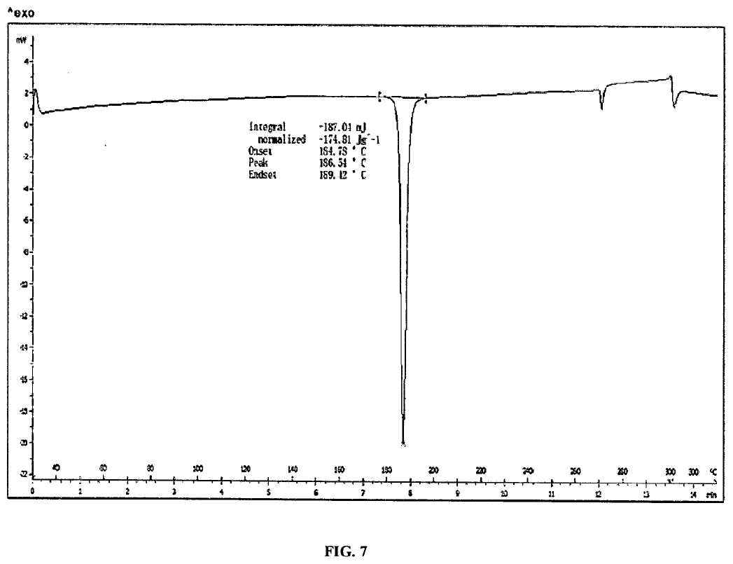

In some embodiments, Crystalline Form I is characterized by an unchanged XRPD after storage at 40° C. and 75% relative humidity (RH) for 6 months. In some embodiments, Crystalline Form I is characterized by an XRPD substantially the same as shown in FIG. 6 after storage at 40° C. and 75% relative humidity (RH) for 6 months. In some embodiments, Crystalline Form I is characterized by an unchanged DSC after storage at 40° C. and 75% relative humidity (RH) for 6 months. In some embodiments, Crystalline Form I is characterized by an DSC substantially the same as shown in FIG. 7 after storage at 40° C. and 75% relative humidity (RH) for 6 months.

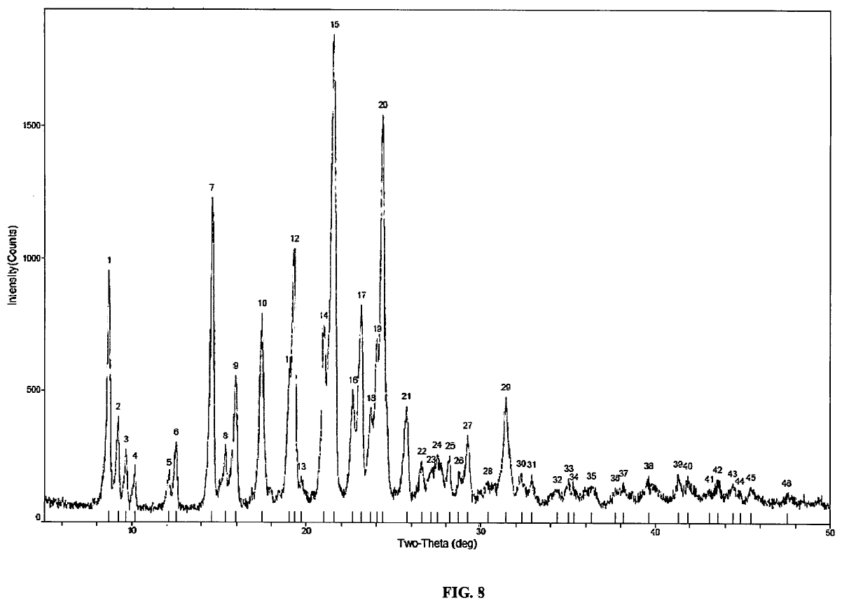

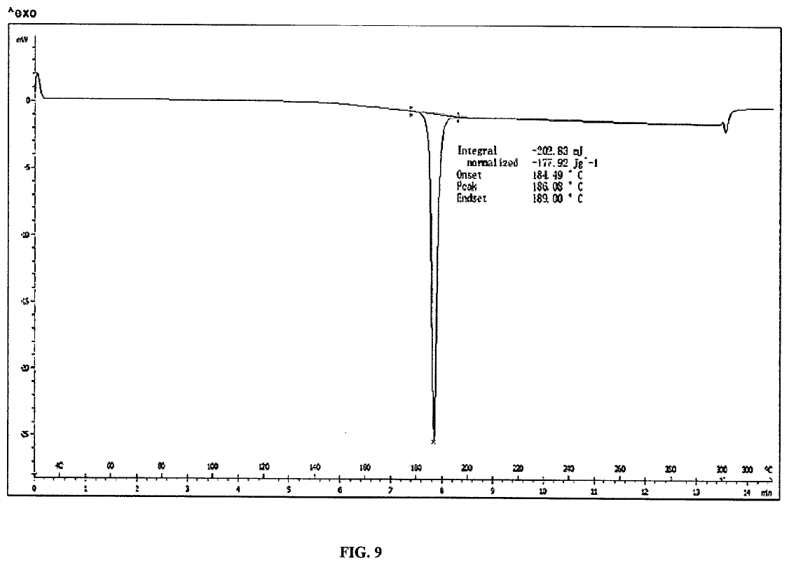

In some embodiments, Crystalline Form I is characterized by an unchanged XRPD after storage at 25° C. and 60% relative humidity (RH) for 6 months. In some embodiments, Crystalline Form I is characterized by an XRPD substantially the same as shown in FIG. 8 after storage at 25° C. and 60% relative humidity (RH) for 6 months. In some embodiments, Crystalline Form I is characterized by an unchanged DSC after storage at 25° C. and 60% relative humidity (RH) for 6 months. In some embodiments, Crystalline Form I is characterized by an DSC substantially the same as shown in FIG. 9 after storage at 25° C. and 60% relative humidity (RH) for 6 months.

In various embodiments, Crystalline Form I is stable at room temperature. In some examples, Crystalline Form I can be stored at room temperature for extended period of time without significant chemical degradation or change in the crystalline form. In some examples, Crystalline Form I can be stored at room temperature for a time period of at least about 10 days, 30 days, 60 days, 90 days, 120 days, 150 days, or 180 days. In some examples, Crystalline Form I can be stored at room temperature for a time period of more than about 180 days. In some examples, Crystalline Form I can be stored at room temperature for a time period of 10-14 days, 10-18 days, 10-22 days, 10-26 days, 10-30 days, 10-40 days, 10-50 days, 10-60 days, 10-90 days, 10-120 days, 10-150 days, 10-180 days, 14-18 days, 14-22 days, 14-26 days, 14-30 days, 14-40 days, 14-50 days, 14-60 days, 14-90 days, 14-120 days, 14-150 days, 14-180 days, 18-22 days, 18-26 days, 18-30 days, 18-40 days, 18-50 days, 18-60 days, 18-90 days, 18-120 days, 18-150 days, 18-180 days, 22-26 days, 22-30 days, 22-40 days, 22-50 days, 22-60 days, 22-90 days, 22-120 days, 22-150 days, 22-180 days, 26-30 days, 26-40 days, 26-50 days, 26-60 days, 26-90 days, 26-120 days, 26-150 days, 26-180 days, 30-40 days, 30-50 days, 30-60 days, 30-90 days, 30-120 days, 30-150 days, 30-180 days, 40-50 days, 40-60 days, 40-90 days, 40-120 days, 40-150 days, 40-180 days, 50-60 days, 50-90 days, 50-120 days, 50-150 days, 50-180 days, 60-90 days, 60-120 days, 60-150 days, 60-180 days, 90-120 days, 90-150 days, or 90-180 days. In some examples, Crystalline Form I can be stored at room temperature for a time period of at least 10 days, 14 days, 18 days, 22 days, 26 days, 30 days, 40 days, 50 days, 60 days, 90 days, 120 days, 150 days, or 180 days.

In various embodiments, Crystalline Form I is stable at temperatures above the room temperature and/or at high RH. In some examples, Crystalline Form I can be stored at about 40° C. at about 75% RH for an extended period of time without significant chemical degradation or change in the crystalline form. In some examples, Crystalline Form I can be stored at 40° C. and at about 75% RH for a time period of at least about 10 days, 30 days, 60 days, 90 days, 120 days, 150 days, or 180 days. In some examples, Crystalline Form I can be stored at 40° C. and at about 75% RH for a time period of more than about 180 days. In some examples, Crystalline Form I can be stored at 40° C. and at about 75% RH for a time period of 10-14 days, 10-18 days, 10-22 days, 10-26 days, 10-30 days, 10-40 days, 10-50 days, 10-60 days, 10-90 days, 10-120 days, 10-150 days, 10-180 days, 14-18 days, 14-22 days, 14-26 days, 14-30 days, 14-40 days, 14-50 days, 14-60 days, 14-90 days, 14-120 days, 14-150 days, 14-180 days, 18-22 days, 18-26 days, 18-30 days, 18-40 days, 18-50 days, 18-60 days, 18-90 days, 18-120 days, 18-150 days, 18-180 days, 22-26 days, 22-30 days, 22-40 days, 22-50 days, 22-60 days, 22-90 days, 22-120 days, 22-150 days, 22-180 days, 26-30 days, 26-40 days, 26-50 days, 26-60 days, 26-90 days, 26-120 days, 26-150 days, 26-180 days, 30-40 days, 30-50 days, 30-60 days, 30-90 days, 30-120 days, 30-150 days, 30-180 days, 40-50 days, 40-60 days, 40-90 days, 40-120 days, 40-150 days, 40-180 days, 50-60 days, 50-90 days, 50-120 days, 50-150 days, 50-180 days, 60-90 days, 60-120 days, 60-150 days, 60-180 days, 90-120 days, 90-150 days, or 90-180 days. In some examples, Crystalline Form I can be stored at 40° C. at about 75% RH for a time period of at least 10 days, 14 days, 18 days, 22 days, 26 days, 30 days, 40 days, 50 days, 60 days, 90 days, 120 days, 150 days, or 180 days.

In various embodiments, Crystalline Form I is stable at temperatures above the room temperature and/or at high RH. In some examples, Crystalline Form I can be stored at about 25° C. at about 60% RH for an extended period of time without significant chemical degradation or change in the crystalline form. In some examples, Crystalline Form I can be stored at 25° C. at about 60% RH for a time period of at least about 10 days, 30 days, 60 days, 90 days, 120 days, 150 days, or 180 days. In some examples, Crystalline Form I can be stored at 25° C. at about 60% RH for a time period of more than about 180 days. In some examples, Crystalline Form I can be stored at 25° C. at about 60% RH for a time period of 10-14 days, 10-18 days, 10-22 days, 10-26 days, 10-30 days, 10-40 days, 10-50 days, 10-60 days, 10-90 days, 10-120 days, 10-150 days, 10-180 days, 14-18 days, 14-22 days, 14-26 days, 14-30 days, 14-40 days, 14-50 days, 14-60 days, 14-90 days, 14-120 days, 14-150 days, 14-180 days, 18-22 days, 18-26 days, 18-30 days, 18-40 days, 18-50 days, 18-60 days, 18-90 days, 18-120 days, 18-150 days, 18-180 days, 22-26 days, 22-30 days, 22-40 days, 22-50 days, 22-60 days, 22-90 days, 22-120 days, 22-150 days, 22-180 days, 26-30 days, 26-40 days, 26-50 days, 26-60 days, 26-90 days, 26-120 days, 26-150 days, 26-180 days, 30-40 days, 30-50 days, 30-60 days, 30-90 days, 30-120 days, 30-150 days, 30-180 days, 40-50 days, 40-60 days, 40-90 days, 40-120 days, 40-150 days, 40-180 days, 50-60 days, 50-90 days, 50-120 days, 50-150 days, 50-180 days, 60-90 days, 60-120 days, 60-150 days, 60-180 days, 90-120 days, 90-150 days, or 90-180 days. In some examples, Crystalline Form I can be stored at 25° C. at about 92.5% RH for a time period of at least 10 days, 14 days, 18 days, 22 days, 26 days, 30 days, 40 days, 50 days, 60 days, 90 days, 120 days, 150 days, or 180 days.

### Crystalline Form II of Compound A

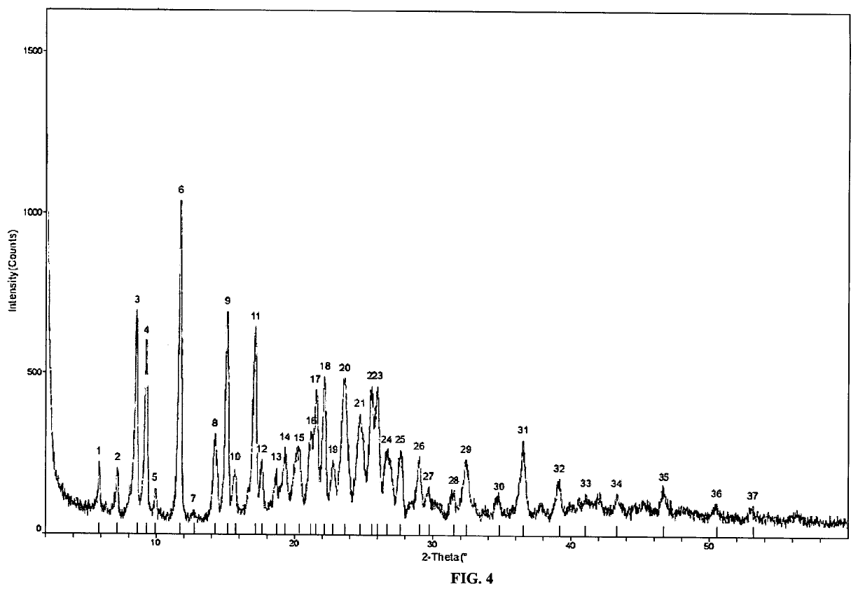

FIG. 4 shows the X-ray powder diffraction (XRPD) pattern for Crystalline Form II of Compound A.

FIG. 5 shows the differential scanning calorimetry (DSC) thermogram for Crystalline Form II of Compound A.

In one aspect, provided herein is Crystalline Form II of N-(2,4,5-trifluoro-3-(3-morpholinoquinoxaline-6-carbonyl)phenyl)propane-1-sulfonamide. Some embodiments provide a composition comprising Crystalline Form II of N-(2,4,5-trifluoro-3-(3-morpholinoquinoxaline-6-carbonyl)phenyl)propane-1-sulfonamide. In some embodiments, Crystalline Form II of N-(2,4,5-trifluoro-3-(3-morpholinoquinoxaline-6-carbonyl)phenyl)propane-1-sulfonamide is characterized as having:
(a) an X-ray powder diffraction pattern comprising peaks at 11.8±0.2° 2-θ, 8.6±0.2° 2-θ, and 15.1±0.2° 2-θ, as measured by X-ray powder diffraction using an X-ray wavelength of 1.5406 Å;
(b) an X-ray powder diffraction pattern substantially the same as shown in FIG. 4;
(c) a differential scanning calorimetry (DSC) thermogram comprising:
   i) an endotherm in the range of about 135-145° C.;
   ii) an exotherm in the range of about 143-153° C.; and
   iii) an endotherm in the range of 155-165° C.;
(d) a differential scanning calorimetry (DSC) thermogram comprising
   i) an endotherm with an onset of about 138° C. and a peak of about 143° C.;
   ii) an exotherm with an onset of about 146° C. and a peak of about 148° C.; and
   iii) an endotherm with an onset of about 158° C. and a peak of about 160° C.;
(e) a differential scanning calorimetry (DSC) thermogram substantially the same as shown in FIG. 5;
   or
(j) combinations thereof.

In some embodiments, Crystalline Form II is characterized by an X-ray powder diffraction pattern substantially the same as shown in FIG. 4.

In some embodiments, Crystalline Form II is characterized by an X-ray powder diffraction pattern comprising peaks at 11.8±0.2° 2-θ, 8.6±0.2° 2-θ, and 15.1±0.2° 2-θ, as measured by X-ray powder diffraction using an X-ray wavelength of 1.5406 Å. In some embodiments, Crystalline Form II is characterized by an X-ray powder diffraction pattern comprising peaks at 11.8±0.1° 2-θ, 8.6±0.1° 2-θ, and 15.1±0.1° 2-θ, as measured by X-ray powder diffraction using an X-ray wavelength of 1.5406 Å. In some embodiments, Crystalline Form II is characterized by an X-ray powder diffraction pattern comprising peaks at about 11.8° 2-θ, about 8.6° 2-θ, and about 15.1° 2-θ, as measured by X-ray powder diffraction using an X-ray wavelength of 1.5406 Å.

In some embodiments, the X-ray powder diffraction pattern further comprises at least one peak selected from 17.2±0.2° 2-θ, 9.3±0.2° 2-θ, and 23.6±0.2° 2-θ, as measured by X-ray powder diffraction using an X-ray wavelength of 1.5406 Å. In some embodiments, the X-ray powder diffraction pattern further comprises at least one peak selected from 17.2±0.1° 2-θ, 9.3±0.1° 2-θ, and 23.6±0.1° 2-θ, as measured by X-ray powder diffraction using an X-ray wavelength of 1.5406 Å. In some embodiments, the X-ray powder diffraction pattern further comprises at least one peak selected from about 17.2° 2-θ, about 9.3° 2-θ, and about 23.6° 2-θ, as measured by X-ray powder diffraction using an X-ray wavelength of 1.5406 Å.

In some embodiments, the X-ray powder diffraction pattern further comprises at least one peak selected from 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern further comprises at least one peak selected from 21.5±0.1° 2-θ, 22.2±0.1° 2-θ, and 14.3±0.1° 2-θ, as measured by X-ray powder diffraction using an X-ray wavelength of 1.5406 Å. In some embodiments, the X-ray powder diffraction pattern further comprises at least one peak selected from about 21.5° 2-θ, about 22.2° 2-θ, and about 14.3° 2-θ, as measured by X-ray powder diffraction using an X-ray wavelength of 1.5406 Å.

In some embodiments, the X-ray powder diffraction pattern comprises a peak selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises at least two peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises at least three peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises at least four peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises at least five peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises at least six peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises at least seven peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises at least eight peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises peaks at 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å. In some embodiments, the X-ray powder diffraction pattern comprises peaks at 11.8±0.1° 2-θ, 8.6±0.1° 2-θ, 15.1±0.1° 2-θ, 17.2±0.1° 2-θ, 9.3±0.1° 2-θ, 23.6±0.1° 2-θ, 21.5±0.1° 2-θ, 22.2±0.1° 2-θ, and 14.3±0.1° 2-θ, as measured by X-ray powder diffraction using an X-ray wavelength of 1.5406 Å. In some embodiments, the X-ray powder diffraction pattern comprises peaks at about 11.8° 2-θ, about 8.6° 2-θ, about 15.1° 2-θ, about 17.2° 2-θ, about 9.3° 2-θ, about 23.6° 2-θ, about 21.5° 2-θ, about 22.2° 2-θ, and about 14.3° 2-θ, as measured by X-ray powder diffraction using an X-ray wavelength of 1.5406 Å.

In some embodiments, Crystalline Form II is characterized by a differential scanning calorimetry (DSC) thermogram substantially the same as shown in FIG. 5. In some embodiments, Crystalline Form II is characterized by a differential scanning calorimetry (DSC) thermogram comprising: an endotherm in the range of about 135-145° C.; an exotherm in the range of about 143-153° C.; and an endotherm in the range of 155-165° C. In some embodiments, Crystalline Form II is characterized by a differential scanning calorimetry (DSC) thermogram comprising: an endotherm with an onset of about 138° C. and a peak of about 143° C.; an exotherm with an onset of about 146° C. and a peak of about 148° C.; and an endotherm with an onset of about 158° C. and a peak of about 160° C.

In some embodiments, the Crystalline Form II is characterized by an endotherm in the range of about 130-160° C., for example at about 130-160° C., 130-155° C., 130-150° C., 130-145° C., 130-140° C., 130-135° C., 135-160° C., 135-155° C., 135-150° C., 135-145° C., 135-140° C., 140-160° C., 140-155° C., 140-150° C., 140-145° C., 145-160° C., 145-155° C., 145-150° C., 150-160° C., 150-155° C., or 155-160° C. in the DSC thermogram. In some examples, the Crystalline Form II is characterized by an endotherm at about 143° C. in the DSC thermogram.

In some embodiments, the Crystalline Form II is characterized by an exotherm in the range of about 135-165° C., for example at about 135-165° C., 135-160° C., 135-155° C., 135-150° C., 135-145° C., 135-140° C., 140-165° C., 140-160° C., 140-155° C., 140-150° C., 140-145° C., 145-165° C., 145-160° C., 145-155° C., 145-150° C., 150-165° C., 150-160° C., 150-155° C., 155-165° C., 155-160° C., or 160-165° C. in the DSC thermogram. In some examples, the Crystalline Form II is characterized by an exotherm at about 148° C. in the DSC thermogram.

In some embodiments, the Crystalline Form II is further characterized by an endotherm in the range of about 150-180° C., for example at about 150-175° C., 150-170° C., 150-165° C., 150-160° C., 150-155° C., 155-180° C., 155-175° C., 155-170° C., 155-165° C., 155-160° C., 160-180° C., 160-175° C., 160-170° C., 160-165° C., 165-180° C., 165-175° C., 165-170° C., 170-180° C., 170-175° C., or 175-180° C. in the DSC thermogram. In some examples, the Crystalline Form II is further characterized by an endotherm at about 160° C. in the DSC thermogram.

### II. Methods of Making Compound A and Polymorphic Forms Thereof

In one aspect, the invention provides methods of making one or more polymorphs of Compound A:

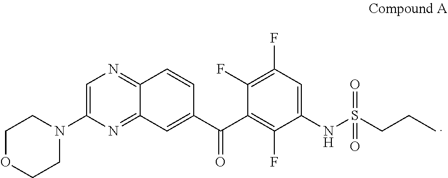 [SMILES: CCCS(=O)(=O)Nc1cc(F)c(F)c(C(=O)c2ccc3ncc(N4CCOCC4)nc3c2)c1F]

Compound A may be prepared as described previously in WO 2013/032951, U.S. Pat. Nos. 9,295,671, 9,572,808, 10,137,125, and 10,561,652. In some embodiments, Compound A is prepared according to the examples herein.

The polymorphs according to the invention are not limited by the starting materials used to produce Compound A.

In one aspect, the invention is directed to methods of making polymorphs of Compound A, or a pharmaceutically acceptable salt and/or solvate thereof, either by isolation of the desired polymorph as the first solid form after synthesis of Compound A, or alternatively, by isolation of the desired polymorph as a transition from a prior solid form of Compound A. Transitions from one form to another are within the scope of the invention because they can be an alternative manufacturing method for obtaining the form desired for the production of the medicinal preparations.

Polymorphs of Compound A, according to the methods of the invention can be selected from Crystalline Form I, Crystalline Form II, and mixtures thereof.

Isolation and purification of the chemical entities and intermediates described herein can be performed, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples below. However, other equivalent separation or isolation procedures can also be used. Prior to crystallization, Compound A may be isolated in about 50% chemical purity, 55% chemical purity, 60% chemical purity, 65% chemical purity, 70% chemical purity, 75% chemical purity, 80% chemical purity, 90% chemical purity, 91% chemical purity, 92% purity, 93% chemical purity, 94% chemical purity, 95% chemical purity, 96% chemical purity, 97% chemical purity, 98% chemical purity, 99% chemical purity, about 98% chemical purity, or about 100% chemical purity.

In some embodiments, the crystalline forms disclosed herein are obtained by crystallizing Compound A with a chemical purity of less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 78%, less than about 76%, less than about 74%, less than about 72%, or less than about 70%. In some embodiments, the crystalline forms are obtained by crystallizing Compound A with a chemical purity in the range of about 70% to about 99%, 80% to about 96%, about 85% to about 96%, about 90% to about 96%, about 80% to 98%, about 85% to about 98%, about 90% to about 98%, about 92% to about 98%, about 94% to 98%, or about 96% to about 98%.

### Preparation of Crystalline Form I

In one embodiment, the desired polymorph is Crystalline Form I of Compound A, and the isolating step involves recrystallization of crude reaction product from a mono-solvent system. In various embodiments, the desired polymorph is Crystalline Form I of Compound A, and the isolating step involves recrystallization of crude product from a binary, tertiary, or greater solvent system, collectively understood as a multi-solvent system. In various embodiments, the desired polymorph is Crystalline Form I of Compound A, and the isolating step involves crystallization from a mono- or multi-solvent system, where the crystallization involves dissolving Compound A in the mono- or multi-solvent system at a temperature above ambient temperature. In some examples, the dissolving of Compound A in the mono- or multi-solvent system is performed at a temperature of about 40-90° C., 45-90° C., 50-90° C., 55-90° C., 60-90° C., 65-90° C., 70-90° C., 75-90° C., 40-85° C., 45-85° C., 50-85° C., 55-85° C., 60-85° C., 65-85° C., 70-85° C., 75-85° C., 80-85° C., 40-80° C., 45-80° C., 50-80° C., 55-80° C., 60-80° C., 65-80° C., 70-80° C., 75-80° C., 40-75° C., 45-75° C., 50-75° C., 55-75° C., 60-75° C., 65-75° C., 70-75° C., 40-70° C., 45-70° C., 50-70° C., 55-70° C., 60-70° C., 65-70° C., 40-65° C., 45-65° C., 50-65° C., 55-65° C., 60-65° C., 40-60° C., 45-60° C., 50-60° C., 55-60° C., 40-55° C., 45-55° C., 50-55° C., 40-50° C., or 45-50° C. In some examples, the recrystallization solvent comprises ethyl acetate and the dissolving of Compound A in the solvent is performed at a temperature of about 75-85° C. Any suitable amount of solvent can be used for dissolving Compound A. In some embodiments, the amount of solvent (e.g., ethyl acetate) used to dissolve the compound is from about 1-10 mL per gram of Compound A. For example, in some embodiments, the amount of solvent used for dissolving Compound A is 4 mL per gram of Compound A. In some examples, the recrystallization solvent comprises ethyl acetate, the dissolving of Compound A in the solvent system is performed at a temperature of about 75-85° C., and the amount of solvent used for dissolving is about 4 mL/g of Compound A.

In various embodiments, the crystallization further involves actively heating the solution containing the dissolved Compound A, for example to a temperature of about 40-100° C., 40-90° C., 40-80° C., 40-70° C., 40-60° C., 40-50° C., 50-100° C., 50-90° C., 50-80° C., 50-70° C., 50-60° C., 60-100° C., 60-90° C., 60-80° C., 60-70° C., 70-100° C., 70-90° C., 70-80° C., 80-100° C., or 80-90° C. In some embodiments, the solution containing the dissolved Compound A, is heated to a temperature of about 75-85° C. In various embodiments, the solution containing the dissolved Compound A is maintained at the heated temperature for a period of time, for example for about 30 min, about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h or more.

In various embodiments, the crystallization further involves actively cooling the heated solution containing the dissolved Compound A, for example to a temperature of about 0-40° C., 0-30° C., 0-20° C., 0-10° C., 10-40° C., 10-30° C., 10-20° C., 20-40° C., 20-30° C., 20-10° C., or 30° C.-40° C. In some embodiments, the crystallization further involves actively cooling the heated solution containing the dissolved Compound A to a temperature of about 20-30° C. In various embodiments, the solution containing the dissolved Compound A is further maintained at this lower temperature for a time period, for example for about 30 min, about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h or more.

In various embodiments, the steps of active heating followed by active cooling are repeated multiple times, for example at least 2, at least 3, at least 4, at least 5, at least 6, at least 7, at least 8, at least 9, or at least 10 times. In some embodiments, the steps of active heating followed by active cooling are repeated 2, 3, 4, 5, 6, 7, 8, 9, or 10 times.

In various embodiments, the crystallization further involves filtering the solution containing the obtained crystals of Compound A. In some embodiments, the crystallization optionally involves washing the obtained crystals by a solvent, for example by the recrystallization solvent one or more times. In some embodiments, the crystallization optionally involves drying the obtained crystals, for example under vacuum at a temperature of about 40-50° C.

In some embodiments, the chemical purity of Crystalline Form I is greater than 60%, 70%, 80%, 90%, 95%, or 99%. In some embodiments, the chemical purity of Crystalline Form I is greater than about 90%. In some embodiments, the chemical purity of Crystalline Form I is greater than about 95%. In some embodiments, the chemical purity of Crystalline Form I greater than about 99%. The chemical purity of Crystalline Form I may be measured by any available analytical technique, for example by HPLC analysis.

In various embodiments, Crystalline Form I is dry. In various embodiments, Crystalline Form I is non-solvated. In various embodiments, Crystalline Form I is non-hydrated. In various embodiments, Crystalline Form I is anhydrous.

### Preparation of Crystalline Form II

In one embodiment, the desired polymorph is Crystalline Form II of Compound A, and the isolating step involves recrystallization of crude reaction product from a mono-solvent system. In various embodiments, the desired polymorph is Crystalline Form II of Compound A, and the isolating step involves recrystallization of crude product from a binary, tertiary, or greater solvent system, collectively understood as a multi-solvent system. In various embodiments, the desired polymorph is Crystalline Form II of Compound A, and the isolating step involves crystallization from a mono- or multi-solvent system. In some examples, the dissolving of Compound A in the mono- or multi-solvent system is performed at a temperature of about 10-50° C., 10-45° C., 10-40° C., 10-35° C., 10-30° C., 10-25° C., 10-20° C., 10-15° C., 15-50° C., 15-45° C., 15-40° C., 15-35° C., 15-30° C., 15-25° C., 15-20° C., 20-50° C., 20-45° C., 20-40° C., 20-35° C., 20-30° C., 20-25° C., 25-50° C., 25-45° C., 25-40° C., 25-35° C., 25-30° C., 30-50° C., 30-45° C., 30-40° C., 30-35° C., 35-50° C., 35-45° C., 35-40° C., 40-50° C., 40-45° C., or 45-50° C. In some examples, the recrystallization solvent comprises ethyl acetate and the dissolving of Compound A in the solvent is performed at a temperature of about 10-20° C. Any suitable amount of solvent can be used for dissolving Compound A. In some embodiments, the amount of solvent (e.g., ethyl acetate) used to dissolve the compound is from about 1-10 mL per gram of Compound A. For example, in some embodiments, the amount of solvent used for dissolving Compound A is 1.5 mL per gram of Compound A. In some examples, the recrystallization solvent comprises ethyl acetate, the dissolving of Compound A in the solvent system is performed at a temperature of about 10-20° C., and the amount of solvent used for dissolving is about 1.5 mL/g of Compound A.

In various embodiments, the crystallization further involves filtering the solution containing the obtained crystals of Compound A. In some embodiments, the crystallization optionally involves washing the obtained crystals by a solvent, for example by the recrystallization solvent one or more times. In some embodiments, the crystallization optionally involves drying the obtained crystals, for example under vacuum at a temperature of about 50-60° C.

In some embodiments, the chemical purity of the Crystalline Form II is greater than 60%, 70%, 80%, 90%, 95%, or 99%. In some embodiments, the chemical purity of the Crystalline Form II is greater than about 90%. In some embodiments, the chemical purity of the Crystalline Form II is greater than about 95%. In some embodiments, the chemical purity of the Crystalline Form II greater than about 99%. The chemical purity of Crystalline Form II may be measured by any available analytical technique, for example by HPLC analysis.

### III. Additional Definitions

As used herein, “active agent” is used to indicate a chemical entity which has biological activity. In certain embodiments, an “active agent” is a compound having pharmaceutical utility. For example an active agent may be an anti-cancer therapeutic.

As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the compound with the target or due to the interaction of the compound with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

As used herein, “therapeutically effective amount” of a chemical entity described herein refers to an amount effective, when administered to a human or non-human subject, to provide a therapeutic benefit such as amelioration of symptoms, slowing of disease progression, or prevention of disease.

“Treating” or “treatment” encompasses administration of Compound A, or a pharmaceutically acceptable salt thereof, to a mammalian subject, particularly a human subject, in need of such an administration and includes (i) arresting the development of clinical symptoms of the disease, such as cancer, (ii) bringing about a regression in the clinical symptoms of the disease, such as cancer, and/or (iii) prophylactic treatment for preventing the onset of the disease, such as cancer.

As used herein, a “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salts” include, but are not limited to salts with inorganic acids, such as hydrochlorate, carbonate, phosphate, hydrogenphosphate, diphosphate, hydrobromate, sulfate, sulfinate, nitrate, and like salts; as well as salts with an organic acid, such as malate, malonate, maleate, fumarate, tartrate, succinate, citrate, acetate, lactate, gluconate, methanesulfonate, Tris (hydroxymethyl-aminomethane), p-toluenesulfonate, priopionate, 2-hydroxyethylsulfonate, benzoate, salicylate, stearate, oxalate, pamoate, and alkanoate such as acetate, HOOC—(CH₂)ₙ—COOH where n is 0-4, and like salts. Other salts include sulfate, methasulfonate, bromide, trifluoracetate, picrate, sorbate, benzilate, salicilate, nitrate, phthalate or morpholine. Pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As used herein, “subject” refers to a mammal that has been or will be the object of treatment, observation or experiment. The methods described herein can be useful in both human therapy and veterinary applications. In some embodiments, the subject is a human.

The term “mammal” is intended to have its standard meaning, and encompasses humans, dogs, cats, sheep, and cows, for example.

“Prodrugs” described herein include any compound that becomes Compound A when administered to a subject, e.g., upon metabolic processing of the prodrug. Similarly, “pharmaceutically acceptable salts” includes “prodrugs” of pharmaceutically acceptable salts. Examples of prodrugs include derivatives of functional groups, such as a carboxylic acid group, in Compound A. Exemplary prodrugs of a carboxylic acid group include, but are not limited to, carboxylic acid esters such as alkyl esters, hydroxyalkyl esters, arylalkyl esters, and aryloxyalkyl esters. Other exemplary prodrugs include lower alkyl esters such as ethyl ester, acyloxyalkyl esters such as pivaloyloxymethyl (POM), glycosides, and ascorbic acid derivatives. Other exemplary prodrugs include amides of carboxylic acids. A discussion of prodrugs is provided in T. Higuchi and V. Stella, Pro-drugs as Novel Delivery Systems, Vol. 14 of the A.C.S. Symposium Series, in Edward B. Roche, ed., Bioreversible Carriers in Drug Design, American Pharmaceutical Association and Pergamon Press, 1987, and in Design of Prodrugs, ed. H. Bundgaard, Elsevier, 1985.

The compounds disclosed herein can be used in different enriched isotopic forms, e.g., enriched in the content of ²H, ³H, ¹¹C, ¹³C and/or ¹⁴C. In one particular embodiment, the compound is deuterated at least one position. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Raj ender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates. Also included are solvates formed with the one or more crystallization solvents.

Pharmaceutically acceptable forms of the compounds recited herein include pharmaceutically acceptable salts, chelates, non-covalent complexes, prodrugs, and mixtures thereof.

A “chelate” is formed by the coordination of a compound to a metal ion at two (or more) points. The term “compound” is intended to include chelates of compounds. Similarly, “pharmaceutically acceptable salts” includes chelates of pharmaceutically acceptable salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound”. Similarly, pharmaceutically acceptable salts include “non-covalent complexes” of pharmaceutically acceptable salts.

When ranges are used herein for physical properties, such as molecular weight, or chemical properties, such as chemical formulae, all combinations and sub combinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range. In some instances of numerical ranges, “about” means±10%.

As used herein, “significant” refers to any detectable change that is statistically significant in a standard parametric test of statistical significance such as Student's T-test, where p<0.05.

As used herein, “cancer” refers to all types of cancer or neoplasm or malignant tumors found in mammals, including carcinomas and sarcomas. Examples of cancer are cancer of the brain, breast, cervix, colon, head & neck, kidney, lung, non-small cell lung, melanoma, mesothelioma, ovary, sarcoma, stomach, uterus and Medulloblastoma.

As used herein, the terms “BRAF”, “B-raf”, “B-Raf” and the like are used interchangeably to refer to the gene or protein product of the gene.

### IV. Methods of Treatment

In some embodiments, the various polymorphs of Compound A bind to a kinase including, but not limited to, Abl, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, Kdr, Kit, Lck, Lyn, MAP2K1, MAP2K2, MAP4K4, MAPKAPK2, Met, Mnk1, MLK1, p38, PDGFRA, PDGFRB, PDPK1, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, and Zap70, including any mutated versions thereof. For example, the polymorphs of Compound A bind to a kinase selected from the group consisting of A-Raf, B-Raf, B-Raf V600E mutant, B-Raf V600E/T5291 mutant, c-Raf-1, Fak, FGFR1, FGFR2, FGFR3, FGFR4, Jnk1, Jnk2, Jnk3, Lck, Lyn, Met, Pim1, Pim2, Pim3, Pyk2, Kdr, Src and Ret, and any mutated versions thereof. In some embodiments, Compound A binds to a kinase selected from the group consisting of A-Raf, B-Raf, B-Raf V600E mutant, B-Raf V600E/T5291 mutant, or c-Raf-1. For example, the polymorphs of Compound A bind to a kinase which is B-Raf or B-Raf V600E mutant. In some embodiments, the polymorphs of Compound A bind to a kinase including, but not limited to, Abl, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, Kdr, Kit, Lck, Lyn, MAP2K1, MAP2K2, MAP4K4, MAPKAPK2, Met, Mnk1, MLK1, p38, PDGFRA, PDGFRB, PDPK1, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, and Zap70, including any mutated versions thereof, with a K_{d} which is lower than 50 μM, 25 μM, 10 μM, 5 μM, or 1 μM as measured in an in vitro assay. For example, the polymorphs of Compound A bind to a kinase selected from the group consisting of A-Raf, B-Raf, B-Raf V600E mutant, B-Raf V600E/T5291 mutant, c-Raf-1, Fak, FGFR1, FGFR2, FGFR3, FGFR4, Jnk1, Jnk2, Jnk3, Lck, Lyn, Met, Pim1, Pim2, Pim3, Pyk2, Kdr, Src and Ret, and any mutated versions thereof with a K_{d} which is lower than 50 μM, 25 μM, 10 μM, 5 μM, or 1 μM as measured in an in vitro assay. In some embodiments, the polymorphs of Compound A bind to a kinase selected from the group consisting of A-Raf, B-Raf, B-Raf V600E mutant, B-Raf V600E/T5291 mutant, or c-Raf-1 with a K_{d} which is lower than 50 μM, 25 μM, 10 μM, 5 μM, or 1 μM as measured in an in vitro assay. For example, the polymorphs of Compound A bind to a kinase which is B-Raf or B-Raf V600E mutant with a K_{d} which is lower than 50 μM, 25 μM, 10 μM, 5 μM, or 1 μM as measured in an in vitro assay.

In some embodiments, the polymorphs of Compound A inhibit a kinase including, but not limited to, Abl, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, Kdr, Kit, Lck, Lyn, MAP2K1, MAP2K2, MAP4K4, MAPKAPK2, Met, Mnk1, MLK1, p38, PDGFRA, PDGFRB, PDPK1, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, and Zap70, including any mutated versions thereof. For example, the polymorphs of Compound A inhibit a kinase selected from the group consisting of A-Raf, B-Raf, B-Raf V600E mutant, B-Raf V600E/T5291 mutant, c-Raf-1, Fak, FGFR1, FGFR2, FGFR3, FGFR4, Jnk1, Jnk2, Jnk3, Lck, Lyn, Met, Pim1, Pim2, Pim3, Pyk2, Kdr, Src and Ret, and any mutated versions thereof. In some embodiments, the polymorphs of Compound A inhibit a kinase selected from the group consisting of A-Raf, B-Raf, B-Raf V600E mutant, B-Raf V600E/T5291 mutant, or c-Raf-1. For example, the polymorphs of Compound A inhibit a kinase which is B-Raf or B-Raf V600E mutant. In some embodiments, the polymorphs of Compound A inhibit a kinase including, but not limited to, Abl, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, Kdr, Kit, Lck, Lyn, MAP2K1, MAP2K2, MAP4K4, MAPKAPK2, Met, Mnk1, MLK1, p38, PDGFRA, PDGFRB, PDPK1, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, and Zap70, including any mutated versions thereof with an IC₅₀ in an in vitro assay of 10 μM, 5 μM, 2 μM, 1 μM, 500 nM, 200 nM, 100 nM, 50 nM, 25 nM, 10 nM, 5 nM or less as ascertained in an in vitro kinase assay. For example, the polymorphs of Compound A inhibit a kinase selected from the group consisting of A-Raf, B-Raf, B-Raf V600E mutant, B-Raf V600E/T5291 mutant, c-Raf-1, Fak, FGFR1, FGFR2, FGFR3, FGFR4, Jnk1, Jnk2, Jnk3, Lck, Lyn, Met, Pim1, Pim2, Pim3, Pyk2, Kdr, Src and Ret, and any mutated versions thereof with an IC 50 in an in vitro assay of 10 μM, 5 μM, 2 μM, 1 μM, 500 nM, 200 nM, 100 nM, 50 nM, 25 nM, 10 nM, 5 nM or less as ascertained in an in vitro kinase assay. In some embodiments, the polymorphs of Compound A inhibit a kinase selected from the group consisting of A-Raf, B-Raf, B-Raf V600E mutant, B-Raf V600E/T5291 mutant, or c-Raf-1 with an IC 50 in an in vitro assay of 10 μM, 5 μM, 2 μM, 1 μM, 500 nM, 200 nM, 100 nM, 50 nM, 25 nM, 10 nM, 5 nM or less as ascertained in an in vitro kinase assay. For example, the polymorphs of Compound A inhibit a kinase which is B-Raf or B-Raf V600E mutant with an IC 50 in an in vitro assay of 10 μM, 5 μM, 2 μM, 1 μM, 500 nM, 200 nM, 100 nM, 50 nM, 25 nM, 10 nM, 5 nM or less as ascertained in an in vitro kinase assay.

In some embodiments, the polymorphs of Compound A inhibit the activity of one or more kinases selected from the group consisting of A-Raf, B-Raf, B-Raf V600E mutant, B-Raf V600E/T5291 mutant, and c-Raf-1 with an IC 50 in an in vitro assay of 1 μM, 500 nM, 200 nM, 100 nM, 50 nM, 25 nM, 10 nM, 5 nM or less as ascertained in an in vitro kinase assay.

In some embodiments, the polymorphs of Compound A selectively inhibit the activity of one or more kinases selected from the group consisting of Abl, Akt1, Akt2, Akt3, ALK, Alk5, A-Raf, B-Raf, Brk, Btk, Cdk2, CDK4, CDK5, CDK6, CHK1, c-Raf-1, Csk, EGFR, EphA1, EphA2, EphB2, EphB4, Erk2, Fak, FGFR1, FGFR2, FGFR3, FGFR4, Flt1, Flt3, Flt4, Fms, Frk, Fyn, Gsk3alpha, Gsk3beta, HCK, Her2/Erbb2, Her4/Erbb4, IGF1R, IKK beta, Irak4, Itk, Jak1, Jak2, Jak3, Jnk1, Jnk2, Jnk3, Kdr, Kit, Lck, Lyn, MAP2K1, MAP2K2, MAP4K4, MAPKAPK2, Met, Mnk1, MLK1, p38, PDGFRA, PDGFRB, PDPK1, Pim1, Pim2, Pim3, PKC alpha, PKC beta, PKC theta, Plk1, Pyk2, ROCK1, ROCK2, Ron, Src, Stk6, Syk, TEC, Tie2, TrkA, TrkB, Yes, and Zap70, including any mutated versions thereof.

For example, the polymorphs of Compound A selectively inhibit the activity of one or more kinases selected from the group consisting of A-Raf, B-Raf, B-Raf V600E mutant, B-Raf V600E/T5291 mutant, c-Raf-1, Fak, FGFR1, FGFR2, FGFR3, FGFR4, Jnk1, Jnk2, Jnk3, Lck, Lyn, Met, Pim1, Pim2, Pim3, Pyk2, Kdr, Src and Ret. In some embodiments, the polymorphs of Compound A selectively inhibit the activity of one or more kinases selected from the group consisting of A-Raf, B-Raf, B-Raf V600E mutant, B-Raf V600E/T5291 mutant, and c-Raf-1.

In some embodiments, the polymorphs of Compound A selectively inhibit the activity of B-Raf or B-Raf V600E mutant relative to one or more kinases selected from the group consisting of ABL1, AKT1 (PKB alpha), AURKB (Aurora B), BLK, BTK, CDK1/cyclin B, CHEK1 (CHK1), CSF1R (FMS), CSNK1G2 (CK1 gamma 2), EGFR (ErbB1), FGFR1, FGR, FLT3, FRAP1 (mTOR), FYN, IGF1R, IKBKB (IKK beta), INSR, KDR (VEGFR2), KIT, LCK, LYN A, MAP2K1 (MEK1), MAP4K5 (KHS1), MAPK1 (ERK2), MAPK14 (p38 alpha), MAPKAPK2, MET (cMet), PDGFRB (PDGFR beta), PIK3CA/PIK3R1 (p110 alpha/p85 alpha) PRKCB2 (PKC beta II), PTK2B (FAK2), PTK6 (Brk), RAF1 (cRAF) Y340D Y341D, RET, RPS6KB1 (p70S6K), SRC, SRMS (Srm), and YES1. In some embodiments, the polymorphs of Compound A selectively inhibit the activity of one or more kinases selected from the group consisting of A-Raf, B-Raf, B-Raf V600E mutant, B-Raf V600E/T5291 mutant, and c-Raf-1 with an IC₅₀ which is ½, ⅓^{rd}, ¼^{th}, ⅕^{th}, 1/7^{th}, 1/10^{th}, 1/15^{th}, 1/20^{th}, 1/25^{th}, 1/30^{th}, 1/40^{th}, 1/50^{th}, 1/100^{th}, 1/150^{th}, 1/200^{th}, 1/300^{th}, 1/400^{th}, 1/500^{th}, 1/1000^{th}, 1/2000^{th} or less than the IC₅₀ for a kinase selected from the group consisting of ABL1, AKT1 (PKB alpha), AURKB (Aurora B), BLK, BTK, CDK1/cyclin B, CHEK1 (CHK1), CSF1R (FMS), CSNK1G2 (CK1 gamma 2), EGFR (ErbB1), FGFR1, FGR, FLT3, FRAP1 (mTOR), FYN, IGF1R, IKBKB (IKK beta), INSR, KDR (VEGFR2), KIT, LCK, LYN A, MAP2K1 (MEK1), MAP4K5 (KHS1), MAPK1 (ERK2), MAPK14 (p38 alpha), MAPKAPK2, MET (cMet), PDGFRB (PDGFR beta), PIK3CA/PIK3R1 (p110 alpha/p85 alpha) PRKCB2 (PKC beta II), PTK2B (FAK2), PTK6 (Brk), RAF1 (cRAF) Y340D Y341D, RET, RPS6KB1 (p70S6K), SRC, SRMS (Srm), and YES1.

In some embodiments, one or more polymorphs of Compound A are capable of inhibiting cellular proliferation. For example, in some cases, one or more polymorphs of Compound A inhibit proliferation of tumor cells or tumor cell lines. For example, such cell lines express a kinase which is B-raf or B-raf V600E mutant. In some cases, one or more polymorphs of Compound A inhibit A375 or SK-MEL-28 cell proliferation in vitro or in an in vivo model such as a xenograft mouse model. In some cases, in vitro cultured A375 or SK-MEL-28 cell proliferation may be inhibited with an IC 50 of less than 100 nM, 75 nM, 50 nM, 25 nM, 15 nM, 10 nM, 5 nM, 3 nM, 2 nM, 1 nM, 0.5 nM, 0.1 nM or less by one or more polymorphs of Compound A.

### V. Methods of Use

The polymorphs described herein are useful in the treatment, or in the preparation of a medicament for the treatment of various disorders. For example, the polymorphs of Compound A are useful as inhibitors of protein kinases. In some embodiments, the polymorphs described herein are inhibitors of one or more kinases. For example, the polymorphs of Compound A are inhibitors of A-Raf, B-Raf, C-Raf and of mutants of such kinases, including the B-Raf V600E mutant. Thus, without wishing to be bound by any particular theory, the polymorphs of Compound A are particularly useful for treating or lessening the severity of a disease, condition, or disorder where activation of one or more kinases, such as Raf kinases, which is implicated in the disease, condition, or disorder. When activation of Raf kinases is implicated in a particular disease, condition, or disorder, the disease, condition, or disorder may also be referred to as “Raf-mediated disease” or disease symptom. Accordingly, in another aspect, the present invention provides a method for treating or lessening the severity of a disease, condition, or disorder where activation or one or more of Raf kinases is implicated in the disease state.

The inhibition of Raf kinases may be assayed in vitro, in vivo, or in a cell line. In vitro assays include assays that determine inhibition of either the phosphorylation activity or ATPase activity of activated Raf kinase. Alternate in vitro assays quantitate the ability of the inhibitor to bind to Raf kinase. Inhibitor binding may be measured by radiolabeling the inhibitor prior to binding, isolating the inhibitor, complex and determining the amount of radiolabel bound. Alternatively, inhibitor binding may be determined by running a competition experiment where new inhibitors are incubated with Raf kinase bound to known radioligands.

The polymorphs described herein may be prepared in substantially pure form, typically by standard chromatographic methods, prior to formulation in a pharmaceutically acceptable form.

The polymorphs described herein may be used in treating a variety of neoplasms, including malignant and benign tumors, as well as cancers. Cancers that can be prevented and/or treated by the polymorphs, compositions, and methods described herein include, but are not limited to, human sarcomas and carcinomas, e.g., carcinomas, e.g., colon carcinoma, pancreatic cancer, breast cancer, ovarian cancer, prostate cancer, thyroid cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, Langerhans cell histiocytosis (LCH), Erdheim-Chester disease (ECD), leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. Benign tumors that can be prevented and/or treated by the polymorphs, compositions, and methods described herein include, but are not limited to, craniopharyngioma.

In some embodiments, the polymorphs described herein are used for the treatment of cancers of the
i. digestive system including, without limitation, the esophagus, stomach, small intestine, colon (including colorectal), liver & intrahepatic bile duct, gallbladder & other biliary, pancreas, and other digestive organs;
ii. respiratory system, including without limitation, larynx, lung & bronchus, and other respiratory organs;
iii. skin;
iv. thyroid;
v. breast;
vi. genital system, including without limitation, uterine cervix, ovary, and prostate;
vii. urinary system, including without limitation, urinary bladder and kidney and renal pelvis; and
viii. oral cavity & pharynx, including without limitation, tongue, mouth, pharynx, and other oral cavity.

In some embodiments, the polymorphs described herein are used for the treatment of colon cancer, liver cancer, lung cancer, melanoma, thyroid cancer, breast cancer, ovarian cancer, and oral cancer.

The polymorphs described herein may also be used in conjunction with other well-known therapeutic agents that are selected for their particular usefulness against the condition that is being treated. For example, the polymorphs described herein may be useful in combination with at least one additional anti-cancer and/or cytotoxic agents. Further, the polymorphs described herein may also be useful in combination with other inhibitors of parts of the signaling pathway that links cell surface growth factor receptors to nuclear signals initiating cellular proliferation.

Such known anti-cancer and/or cytotoxic agents that may be used in combination with the polymorphs described herein include:
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumor antibiotics (for example anthracyclines like adriamycin, bleomycin, doxorubicin, daunomycin, epirubicin, idarubicin, mitomycinC, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3methylenedioxyanilino)-7-[2-(4-methylpiperazin-1-yl)ethoxy]-5-tetrahydropyran-4yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4ylamino}thiazole-5-carboxamide (dasatinib, BMS-354825; J. Med. Chern., 2004, 47, 66586661) and bosutinib (SK1-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB 1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (RI15777) and lonafarnib (SCH66336)), inhibitors of cell signalling through MEK and/or AKT kinases, c-kit inhibitors, abl kinase inhibitors, P13 kinase inhibitors, Plt3 kinase inhibitors, CSF-IR kinase inhibitors, IGF receptor (insulin like growth factor) kinase inhibitors; aurora kinase inhibitors (for example AZD1152, PH739358, VX-680, MLN8054, R763, MP235, MP529, VX-528 and AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU1l248), axitinib (AG-013736), pazopanib (GW 786034) and 4·{4-fluoro-2-methylindol-5-yloxy)-6-methoxy-7-(3pyrrolidin-1-ylpropoxy)quinazoline (AZD2171; Example 240 within WO 00/47212), compounds such as those disclosed in International Patent Applications WO 97/22596, WO 97/30035, WO 97/32856 and WO 98/13354 and compounds that work by other mechanisms (for example linomide, inhibitors of integrin av˜3 function and angiostatin));
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme prodrug therapy) approaches such as those using cytosine deaminase, thymidine kinase or a bacterial nitroreductase enzyme and approaches to increase subject tolerance to chemotherapy or radiotherapy such as multi-drug resistance gene therapy; and
(x) immunotherapy approaches, including for example ex-vivo and in-vivo approaches to increase the immunogenicity of subject's tumor cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumor cell lines and approaches using anti-idiotypic antibodies.

In certain embodiments, the at least one polymorph of Compound A is administered in combination with one or more agents chosen from pacliataxel, bortezomib, dacarbazine, gemcitabine, trastuzumab, bevacizumab, capecitabine, docetaxel, erlotinib, aromatase inhibitors, such as AROMASIN™ (exemestane), and estrogen receptor inhibitors, such as FASLODEX™ (fulvestrant).

When a polymorph of Compound A is administered into a human subject, the daily dosage will normally be determined by the prescribing physician with the dosage generally varying according to the age, weight, and response of the individual subject, as well as the severity of the subject's symptoms.

In one exemplary application, a suitable amount of at least one polymorph of Compound A is administered to a mammal undergoing treatment for cancer, for example, breast cancer. Administration typically occurs in an amount of between about 0.01 mg/kg of body weight to about 100 mg/kg of body weight per day (administered in single or divided doses), such as at least about 0.1 mg/kg of body weight per day. A particular therapeutic dosage can include, e.g., from about 0.01 mg to about 1000 mg of the polymorph of Compound A, such as including, e.g., from about 1 mg to about 1000 mg. The quantity of the at least one polymorph of Compound A in a unit dose of preparation may be varied or adjusted from about 0.1 mg to 1000 mg, such as from about 1 mg to 300 mg, for example 10 mg to 200 mg, according to the particular application. The amount administered will vary depending on the particular IC₅₀ value of the at least one polymorph of Compound A used and the judgment of the attending clinician taking into consideration factors such as health, weight, and age. In combinational applications in which the at least one polymorph of Compound A described herein is not the sole active ingredient, it may be possible to administer lesser amounts of the at least one polymorph of Compound A and still have therapeutic or prophylactic effect.

In some embodiments, the pharmaceutical preparation is in unit dosage form. In such form, the preparation is subdivided into unit doses containing appropriate quantities of the polymorph of Compound A, e.g., an effective amount to achieve the desired purpose.

The actual dosage employed may be varied depending upon the requirements of the subject and the severity of the condition being treated. Determination of the proper dosage for a particular situation is within the skill of the art. Generally, treatment is initiated with smaller dosages which are less than the optimum dose of the at least one polymorph of Compound A. Thereafter, the dosage is increased by small amounts until the optimum effect under the circumstances is reached. For convenience, the total daily dosage may be divided and administered in portions during the day if desired.

The amount and frequency of administration of the at least one polymorph of Compound A, and if applicable other chemotherapeutic agents and/or radiation therapy, will be regulated according to the judgment of the attending clinician (physician) considering such factors as age, condition and size of the subject as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the subject, and in view of the observed responses of the disease to the administered therapeutic agents.

Also, in general, the at least one polymorph of Compound A need not be administered in the same pharmaceutical composition as a chemotherapeutic agent, and may, because of different physical and chemical characteristics, be administered by a different route. For example, the polymorphs/compositions may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of polymorph (and where appropriate, chemotherapeutic agent and/or radiation) will depend upon the diagnosis of the attending physicians and their judgment of the condition of the subject and the appropriate treatment protocol.

The one or more polymorphs of Compound A (and where appropriate chemotherapeutic agent and/or radiation) may be administered concurrently (e.g., simultaneously, essentially simultaneously or within the same treatment protocol) or sequentially, depending upon the nature of the proliferative disease, the condition of the subject, and the actual choice of chemotherapeutic agent and/or radiation to be administered in conjunction (i.e., within a single treatment protocol) with the one or more polymorphs/composition.

In combinational applications and uses, the one or more polymorphs/composition and the chemotherapeutic agent and/or radiation need not be administered simultaneously or essentially simultaneously, and the initial order of administration of the one or more polymorphs/composition, and the chemotherapeutic agent and/or radiation, may not be important. Thus, the at least one polymorph of Compound A may be administered first followed by the administration of the chemotherapeutic agent and/or radiation; or the chemotherapeutic agent and/or radiation may be administered first followed by the administration of the at least one polymorph of Compound A. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject. For example, the chemotherapeutic agent and/or radiation may be administered first, and then the treatment continued with the administration of the at least one polymorph of Compound A followed, where determined advantageous, by the administration of the chemotherapeutic agent and/or radiation, and so on until the treatment protocol is complete.

Thus, in accordance with experience and knowledge, the practicing physician can modify each protocol for the administration of a polymorph of Compound A/composition for treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MM scan, and successive measurements can be used to judge whether or not growth of the tumor has been retarded or even reversed. Relief of disease-related symptoms such as pain, and improvement in overall condition can also be used to help judge effectiveness of treatment.

### VI. Compositions and Formulations

The disclosure provides compositions, including pharmaceutical compositions, comprising one or more crystalline forms of the present invention.

In various embodiments, the ratio of desired crystalline form such as Crystalline Form 1 to all other crystalline forms in a composition is greater than about 1:1, 2:1, 3:1, 4:1, 5:1, 6:1, 7:1, 8:1, 9:1, or more w/w. In other embodiments, the ratio of Crystalline Form II to all other polymorphs is greater than about 1:1, 2:1, 3:1, 4:1, 5:1, 6:1, 7:1, 8:1, 9:1, or more w/w.

In some embodiments, the one or more polymorphs of Compound A are formulated into pharmaceutical compositions. In specific embodiments, pharmaceutical compositions are formulated in a conventional manner using one or more physiologically acceptable carriers comprising excipients and auxiliaries which facilitate processing of the active compounds/polymorphs into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more polymorphs of Compound A and a pharmaceutically acceptable diluent(s), excipient(s), or carrier(s). In certain embodiments, the one or more polymorphs of Compound A are administered as pharmaceutical compositions in which the one or more polymorphs, are mixed with other active ingredients, as in combination therapy. Encompassed herein are all combinations of actives set forth in the combination therapies section below and throughout this disclosure. In specific embodiments, the pharmaceutical compositions include one or more polymorphs of Compound A.

A pharmaceutical composition, as used herein, refers to a mixture of one or more polymorphs of Compound A with other chemical components, such as carriers, stabilizers, diluents, dispersing agents, suspending agents, thickening agents, and/or excipients. In certain embodiments, the pharmaceutical composition facilitates administration of the polymorphs to an organism. In some embodiments, in practicing the methods of treatment or use provided herein, therapeutically effective amounts of one or more polymorphs of Compound A are administered in a pharmaceutical composition to a mammal having a disease or condition to be treated. In specific embodiments, the mammal is a human. In certain embodiments, therapeutically effective amounts vary depending on the severity of the disease, the age and relative health of the subject and other factors. The one or more polymorphs of Compound A described herein are used singly or in combination with one or more therapeutic agents as components of mixtures.

In one embodiment, one or more polymorphs of Compound A are formulated in an aqueous solution. In specific embodiments, the aqueous solution is selected from, by way of example only, a physiologically compatible buffer, such as Hank's solution, Ringer's solution, or physiological saline buffer. In other embodiments, one or more polymorphs of Compound A are formulated for transmucosal administration. In specific embodiments, transmucosal formulations include penetrants that are appropriate to the barrier to be permeated. In still other embodiments wherein the one or more polymorphs described herein are formulated for other parenteral injections, appropriate formulations include aqueous or nonaqueous solutions. In specific embodiments, such solutions include physiologically compatible buffers and/or excipients.

In another embodiment, the polymorphs described herein are formulated for oral administration. The polymorphs of Compound A are formulated by combining the polymorphs with, e.g., pharmaceutically acceptable carriers or excipients. In various embodiments, the polymorphs described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the polymorphs described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the polymorphs described herein is formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the polymorphs described herein are formulated for buccal or sublingual administration. Formulations suitable for buccal or sublingual administration include, by way of example only, tablets, lozenges, or gels. In still other embodiments, the polymorphs described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical composition of a polymorph of Compound A is formulated in a form suitable for parenteral injection as sterile suspension, solution or emulsion in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active polymorphs in water-soluble form. In additional embodiments, suspensions of the active polymorphs are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the polymorphs to allow for the preparation of highly concentrated solutions. Alternatively, in other embodiments, the active ingredient is in powder form for constitution with a suitable vehicle, e.g., sterile pyrogen-free water, before use.

In still other embodiments, the one or more polymorphs of Compound A are administered topically. The one or more polymorphs described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the one or more polymorphs of Compound A are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the one or more polymorphs of Compound A is accomplished by means of iontophoretic patches and the like. In certain embodiments, transdermal patches provide controlled delivery of the one or more polymorphs of Compound A. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the one or more polymorphs of Compound A are formulated for administration by inhalation. Various forms suitable for administration by inhalation include, but are not limited to, aerosols, mists or powders. Pharmaceutical compositions of the polymorphs of Compound A are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator are formulated containing a powder mix of the compound and a suitable powder base such as lactose or starch.

In still other embodiments, the one or more polymorphs of Compound A are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active polymorphs into preparations which can be used pharmaceutically. Proper formulation is dependent upon the route of administration chosen. Any pharmaceutically acceptable techniques, carriers, and excipients are optionally used as suitable. Pharmaceutical compositions comprising the one or more polymorphs of Compound A are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one polymorph of Compound A described herein as an active ingredient. The active ingredient is in free-acid or free-base form, or in a pharmaceutically acceptable salt form. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions, comprising the one or more polymorphs of Compound A described herein include formulating the polymorphs with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, a pharmaceutical composition comprising at least one polymorph of Compound A illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspension contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a polymorph of Compound A. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the polymorphs described herein are delivered using a sustained-release system, such as semipermeable matrices of solid hydrophobic polymers containing the therapeutic agent. Various sustained-release materials are useful herein. In some embodiments, sustained-release capsules release the polymorphs for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### VII. Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a polymorph of Compound A is administered in a local rather than systemic manner, for example, via injection of the polymorph directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, a polymorph of Compound A is provided in the form of a rapid release formulation, in the form of an extended release formulation, or in the form of an intermediate release formulation. In yet other embodiments, a polymorph of Compound A is administered topically.

### VIII. Kits/Articles of Manufacture

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products Include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more polymorphs described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical composition is presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a polymorph of Compound A formulated in a compatible pharmaceutical carrier are prepared, placed in an appropriate container, and labeled for treatment of an indicated condition.

### EXAMPLES

The following examples serve to more fully describe the manner of using the invention. These examples are presented for illustrative purposes and should not serve to limit the true scope of the invention.

In carrying out the procedures of the methods described herein, it is of course to be understood that references to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

### Example 1: Preparation of N-(2,4,5-trifluoro-3-(3-morpholinoquinoxaline-6-carbonyl)phenyl)propane-1-sulfonamide (Compound A)

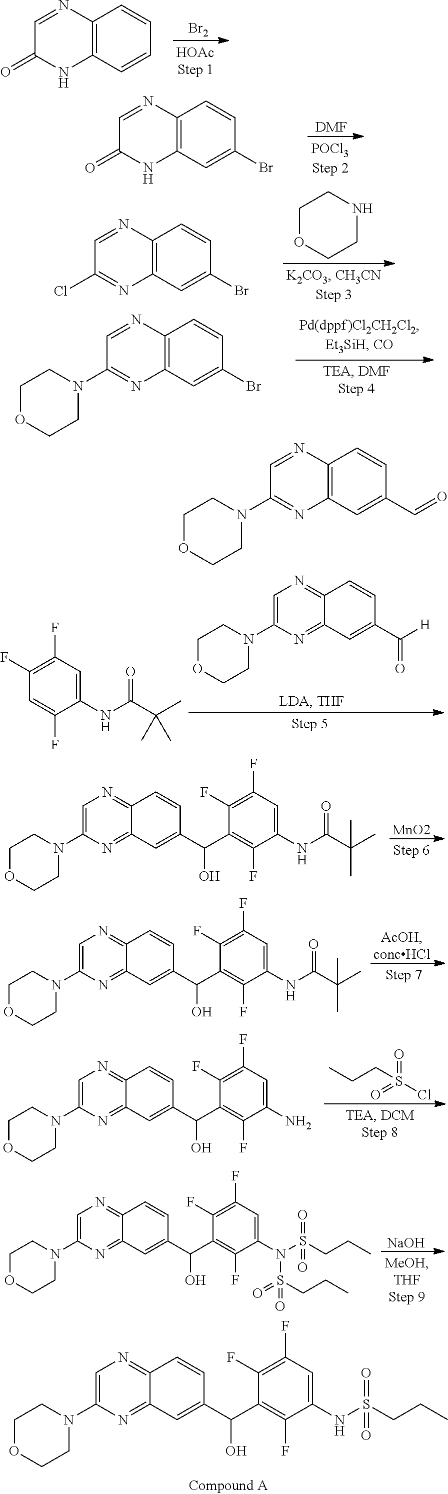

Step 1: To a solution of quinoxalin-2(1H)-one (54.64 g, 374 mmol, 1.0 eq.) in HOAc (1000 mL) was added a solution of Br 2 (19.18 mL, 374 mmol, 1.0 eq.) in HOAc (200 mL) dropwise. The resulting mixture was stirred at rt for 12 h, then poured into ice-water. The precipitate was collected by filtration and dried to afford 7-bromoquinoxalin-2(1H)-one as an off-white solid (74 g, 88%).

Step 2: To a suspension of 7-bromoquinoxalin-2(1H)-one (224 g, 1 mol, 1.0 eq.) in POCl₃ (1000 mL) was added DMF (3.65 g, 0.05 mol, 0.05 eq.). The resulting mixture was stirred at 120° C. for 2 h, then cooled to rt and slowly poured into ice-water with vigorous stirring. The precipitate was collected by filtration and dried to afford 7-bromo-2-chloroquinoxaline as brown solid (180 g, 75%).

Step 3: To a solution of 7-bromo-2-chloroquinoxaline (50 g, 0.2 mol, 1.0 eq.) in CH 3 CN (200 mL) were added morpholine (89 g, 1.02 mol, 5.0 eq.) and K₂CO₃ (85 g, 0.61 mol, 3.0 eq). The resulting mixture was stirred at 90° C. for 2 h, then cooled and filtered. The filtrate was concentrated and the residue was re-crystallized from EA to afford 4-(7-bromoquinoxalin-2-yl)morpholine (59 g, 98.3%).

Step 4: To a solution of 4-(7-bromoquinoxalin-2-yl)morpholine (59 g, 0.2 mol, 1.0 eq.) in DMF (500 mL) was added TEA (139 mL, 1.0 mol, 5.0 eq.), Et₃SiH (127 mL, 0.8 mol, 4.0 eq) and Pd(dppf)Cl₂·CH₂Cl₂ (8.16 g, 0.01 mol, 0.05 eq.). The resulting mixture was stirred at 90° C. for 12 h in an autoclave under CO (1 MPa), then cooled and concentrated. The resulting residue was purified by flash column chromatography (EA/PE=1/1) to afford 3-morpholinoquinoxaline-6-carbaldehyde as a yellow solid (40 g, 82.3%).

Step 5: To a solution of N-(2,4,5-trifluorophenyl)pivalamide (550 mg, 2.4 mmol, 1.2 eq.) in THF (30 mL) cooled at −78° C. was added LDA (4.1 mL, 4.8 mmol, 2.4 eq.) dropwise. The resulting mixture was stirred at −78° C. for 1 h, then a solution of 3-morpholinoquinoxaline-6-carbaldehyde (486 mg, 2.0 mol, 1.0 eq.) in THF (20 mL) was added dropwise. The resulting mixture was stirred at −78° C. for 1 h, then quenched by the addition of NH₄Cl solution. The mixture was extracted with EA (20 mL×3) and the combined organic layers were dried over Na₂SO₄ and concentrated. The resulting residue was purified by flash column chromatography (MeOH/DCM=1/50, v/v) to afford N-(2,4,5-trifluoro-3-(hydroxy(3-morpholinoquinoxalin-6-yl)methyl)phenyl)pivalamide (620 mg, 65.2%).

Step 6: The solution of N-(2,4,5-trifluoro-3-(hydroxy(3-morpholinoquinoxalin-6-yl)methyl)phenyl)pivalamide (620 mg, 1.3 mmol, 1.0 eq.) in DCM (10 mL) was added MnO₂ (358 mg, 6.5 mmol, 5.0 eq.). The resulting mixture was stirred at 50° C. overnight, then cooled and filtered. The filtrate was concentrated and the residue was purified by flash column chromatography (PE/EA=1/2, v/v) to afford N-(2,4,5-trifluoro-3-(3-morpholinoquinoxaline-6-carbonyl)phenyl)pivalamide (560 mg, 90%).

Step 7: To a solution of N-(2,4,5-trifluoro-3-(3-morpholinoquinoxaline-6-carbonyl)phenyl)pivalamide (560 mg, 1.2 mmol, 1.0 eq.) in HOAc (10 mL) was added conc. HCl (50 mL). The mixture was stirred at 110° C. for 4 h, then poured onto ice. The mixture was adjusted to pH 10 by the addition of 1N NaOH solution, then extracted with DCM (100 mL×3). The combined organic layers were dried over Na₂SO₄ and concentrated. The resulting residue was purified by flash column chromatography (PE/EA=1/4, v/v) to afford (3-amino-2,5,6-trifluorophenyl)(3-morpholinoquinoxalin-6-yl)methanone as brown solid (410 mg, 88% yield).

Step 8: To a solution of (3-amino-2,5,6-trifluorophenyl)(3-morpholinoquinoxalin-6-yl)methanone (40 mg, 0.1 mmol, 1.0 eq.) in DCM (10 mL) was added TEA (101 mg, 1 mol, 10 eq.) and propane-1-sulfonyl chloride (0.5 mL, 0.5 mmol, 5.0 eq.). The resulting mixture was stirred at rt for 1 h, then washed with water and extracted with DCM (10 mL×3). The combined organic layers were dried over Na2SO4, filtered and concentrated. The resulting residue was purified by flash column chromatography (PE/EA=2/1, v/v) to afford N-(propylsulfonyl)-N-(2,4,5-trifluoro-3-(3-morpholinoquinoxaline-6-carbonyl)phenyl)propane-1-sulfonamide (41 mg, 62.2%).

Step 9: To a solution of N-(propylsulfonyl)-N-(2,4,5-trifluoro-3-(3-morpholinoquinoxaline-6-carbonyl)phenyl)propane-1-sulfonamide (41 mg, 0.068 mmol, 1.0 eq.) in MeOH/THF (10 mL/10 mL) was added 1 N NaOH (0.15 mmol, 2.2 eq.). The resulting mixture was stirred at rt for 1 h, then concentrated. The resulting residue was purified by flash column chromatography (PE/EA=1/1, v/v) to afford N-(2,4,5-trifluoro-3-(3-morpholinoquinoxaline-6-carbonyl)phenyl)propane-1-sulfonamide (Compound A) (23 mg, 68.9%). LRMS (M+H⁺) m/z calculated 495.1, found 495.1. ¹H NMR (CDCl₃, 400 MHz) δ 8.67 (s, 1H), 7.98-8.03 (m, 3H), 7.66-7.73 (m, 1H), 6.72 (s, 1H), 3.78-3.88 (m, 8H), 3.12-3.16 (t, 2H), 1.87-1.92 (q, 2H), 1.05-1.09 (t, 3H).

### Example 2. Preparation of Crystalline Form I of Compound A

N-(2,4,5-trifluoro-3-(3-morpholinoquinoxaline-6-carbonyl)phenyl)propane-1-sulfonamide (2.53 kg) and ethyl acetate (EA) (9.1 kg) were added to the reactor. The mixture was stirred under refluxing for 2 h. The solution was cooled to room temperature. The resulting precipitate was filtered, washed with EA (1 kg), and dried under vacuum at 45° C. to afford Crystalline Form I of N-(2,4,5-trifluoro-3-(3-morpholinoquinoxaline-6-carbonyl)phenyl)propane-1-sulfonamide (1.94 kg, 76.7%).

### Example 3. Preparation of Crystalline Form II of Compound A

N-(2,4,5-trifluoro-3-(3-morpholinoquinoxaline-6-carbonyl)phenyl)propane-1-sulfonamide (4.01 kg) was dissolved in EA (60 kg), and water (20 kg) was added. The organic phase was separated and concentrated to 4-6 kg under vacuum at 40-45° C. The resulting residue was dissolved in EA (6 kg) and stirred for 4 hours at 10-20° C. The solid was filtered, washed with EA (1.5 kg), and dried under vacuum at 50-55° C. to afford Crystalline Form II of N-(2,4,5-trifluoro-3-(3-morpholinoquinoxaline-6-carbonyl)phenyl)propane-1-sulfonamide (3.15 kg, 78.6%).

### Example 4. X-Ray Powder Diffraction (XRPD)

X-ray powder diffraction (XRPD) patterns were obtained on a Shimadzu XRD-6000. A CuK source (=1.54056 angstrom) operating minimally at 40 kV and 30 mA scans each sample between 5 and 50 degrees 2-theta. The step size is 0.02 degrees 2-theta and the scan speed is 1 second per step.

The XRPD pattern obtained for Crystalline Form I of Compound A is summarized in Table 1 and FIG. 1.

TABLE 1

| Peak # | Angle (°2-θ) | Intensity (%) |
|---|---|---|
| 1 | 8.719 | 100.0 |
| 2 | 9.179 | 9.0 |
| 3 | 9.660 | 14.1 |
| 4 | 10.140 | 3.0 |
| 5 | 12.119 | 6.1 |
| 6 | 12.500 | 7.7 |
| 7 | 14.600 | 45.6 |
| 8 | 15.341 | 6.0 |
| 9 | 15.961 | 22.7 |
| 10 | 17.459 | 53.6 |
| 11 | 17.941 | 4.0 |
| 12 | 19.280 | 35.0 |
| 13 | 19.702 | 3.1 |
| 14 | 21.000 | 31.2 |
| 15 | 21.580 | 85.5 |
| 16 | 22.659 | 13.7 |
| 17 | 23.100 | 23.9 |
| 18 | 23.681 | 10.8 |
| 19 | 24.360 | 60.9 |
| 20 | 25.719 | 14.6 |
| 21 | 26.639 | 7.2 |
| 22 | 27.540 | 5.1 |
| 23 | 28.162 | 2.9 |
| 24 | 29.260 | 12.1 |
| 25 | 30.300 | 1.6 |
| 26 | 31.420 | 16.5 |
| 27 | 32.180 | 3.3 |
| 28 | 32.881 | 2.4 |
| 29 | 34.362 | 1.5 |
| 30 | 34.941 | 2.2 |
| 31 | 36.399 | 3.5 |
| 32 | 38.160 | 2.2 |
| 33 | 38.524 | 0.8 |
| 34 | 39.524 | 2.4 |
| 35 | 41.380 | 3.2 |
| 36 | 41.821 | 3.5 |
| 37 | 43.498 | 2.4 |
| 38 | 44.363 | 2.8 |
| 39 | 45.417 | 2.2 |
| 40 | 47.579 | 1.4 |

The XRPD pattern obtained for Crystalline Form II of Compound A is summarized in Table 2 and FIG. 4.

TABLE 2

| Peak # | Angle (°2-θ) | Intensity (%) |
|---|---|---|
| 1 | 5.863 | 14.7 |
| 2 | 7.180 | 14.1 |
| 3 | 8.576 | 63.5 |
| 4 | 9.280 | 51.5 |
| 5 | 9.930 | 6.9 |
| 6 | 11.759 | 100.0 |
| 7 | 12.737 | 3.0 |
| 8 | 14.279 | 23.9 |
| 9 | 15.140 | 61.9 |
| 10 | 15.718 | 10.2 |
| 11 | 17.179 | 57.5 |
| 12 | 17.658 | 12.3 |
| 13 | 18.678 | 11.3 |
| 14 | 19.300 | 16.2 |
| 15 | 20.340 | 15.8 |
| 16 | 21.200 | 17.4 |
| 17 | 21.521 | 33.6 |
| 18 | 22.161 | 33.0 |
| 19 | 22.780 | 9.6 |
| 20 | 23.640 | 36.0 |
| 21 | 24.741 | 22.2 |
| 22 | 25.599 | 29.3 |
| 23 | 26.000 | 30.2 |
| 24 | 26.660 | 12.3 |
| 25 | 27.661 | 17.2 |
| 26 | 29.039 | 16.0 |
| 27 | 29.721 | 6.3 |
| 28 | 31.539 | 5.4 |
| 29 | 32.398 | 15.7 |
| 30 | 34.795 | 5.4 |
| 31 | 36.518 | 22.9 |
| 32 | 39.159 | 10.8 |
| 33 | 41.057 | 3.9 |
| 34 | 43.303 | 5.3 |
| 35 | 46.702 | 8.0 |
| 36 | 50.541 | 4.1 |
| 37 | 53.137 | 4.4 |

### Example 5. Thermogravimetric Analysis (TGA)

Thermogravimetric analysis was carried out on a TA Instrument TGA unit (NETZSCH TG 209). Samples were heated in platinum pans from ambient to 350° C. at 10 K/min. The TGA thermogram obtained for Crystalline Form I of Compound A is summarized in FIG. 3.

### Example 6. Differential Scanning Calorimetry (DSC)

Differential scanning calorimetry analysis was carried out on a TA Instrument DSC unit (METTLER DSC 3). Samples were heated in non-hermetic aluminum pans from ambient to 300° C. at 20 K/min with a nitrogen purge of 50 mL/min. The DSC thermogram obtained for Crystalline Form I of Compound A is summarized in FIG. 2. The DSC thermogram obtained for Crystalline Form II of Compound A is summarized in FIG. 5.

### Example 7. High-Performance Liquid Chromatography (HPLC)

High-performance liquid chromatography (HPLC) was performed using the following instrument and/or conditions.

| Column: | Agilent extend C18, 150 × 4.6 mm, 3.5 μm | | |
|---|---|---|---|
| Column | 30° C. | | |
| temperature: | | | |
| Detection: | UV at 290 nm | | |
| Injection | 10 μL | | |
| volume: | | | |
| Flow rate: | 1.0 mL/min | | |
| Run time: | 65 min | | |
| Mobile | 10 mmol/L ammonium acetate (adjust pH to 4.0 with | | |
| Phase A: | acetic acid) | | |
| Mobile | Methanol | | |
| Phase B: | | | |
| | Time (minutes) | Mobile Phase A (%) | Mobile Phase B (%) |
| Gradient | 0 | 52 | 48 |
| | 5 | 45 | 55 |
| | 25 | 42 | 58 |
| | 40 | 30 | 70 |
| | 55 | 30 | 70 |
| | 56 | 52 | 48 |
| | 65 | 52 | 48 |

### Example 8. Stability Tests of Crystalline Form I

Crystalline Form I was not changed after storage at 40° C./75% RH for 6 months, or storage at 25° C./60% RH for 6 months. The results of Crystalline Form I in accelerated and long-term stability tests are shown in Table 3 and FIGS. 6-9.

TABLE 3

| | Time | | | | |
|---|---|---|---|---|---|
| Conditions | points | Appearance | Purity | DSC | XRPD |
| 40° C./ | 0 month | Yellow powder | 99.93% | 186.56° C. | Form I |
| 75% RH | 6 months | Yellow powder | 99.92% | 186.54° C. | Form I |
| (closed) | | | | | |
| 25° C./ | 0 month | Yellow powder | 99.93% | 186.56° C. | Form I |
| 60% RH | 6 months | Yellow powder | 99.92% | 186.08° C. | Form I |
| (closed) | | | | | |

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

What is claimed is:

1. Crystalline N-(2,4,5-trifluoro-3-(3-morpholinoquinoxaline-6-carbonyl)phenyl)propane-1-sulfonamide (Compound A): 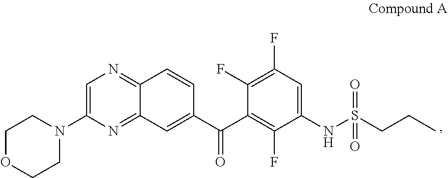 [SMILES: CCCS(=O)(=O)Nc1cc(F)c(F)c(C(=O)c2ccc3ncc(N4CCOCC4)nc3c2)c1F]
or pharmaceutically acceptable solvate or hydrate thereof.

2. The crystalline N-(2,4,5-trifluoro-3-(3-morpholinoquinoxaline-6-carbonyl)phenyl)propane-1-sulfonamide of claim 1, wherein the crystalline form is Crystalline Form I.

3. The crystalline N-(2,4,5-trifluoro-3-(3-morpholinoquinoxaline-6-carbonyl)phenyl)propane-1-sulfonamide of claim 2, wherein Crystalline Form I is characterized by:
(a) an X-ray powder diffraction pattern comprising peaks at 8.7±0.2° 2-θ, 21.6±0.2° 2-θ, and 24.4±0.2° 2-θ, as measured by X-ray powder diffraction using an X-ray wavelength of 1.5406 Å;
(b) an X-ray powder diffraction pattern substantially the same as shown in FIG. 1;
(c) a differential scanning calorimetry (DSC) thermogram comprising an endotherm in the range of about 180-190° C.;
(d) a differential scanning calorimetry (DSC) thermogram comprising an endotherm with an onset of about 184° C. and a peak of about 187° C.;
(e) a differential scanning calorimetry (DSC) thermogram substantially the same as shown in FIG. 2;
(f) a Thermogravimetric Analysis (TGA) thermogram substantially the same as shown in FIG. 3;
(g) an unchanged XRPD after storage at 40° C. and 75% relative humidity (RH) for 6 months;
(h) an unchanged XRPD after storage at 25° C. and 60% relative humidity (RH) for 6 months;
or
(i) combinations thereof.

4. The crystalline N-(2,4,5-trifluoro-3-(3-morpholinoquinoxaline-6-carbonyl)phenyl)propane-1-sulfonamide of claim 2 or claim 3, wherein Crystalline Form I is characterized by an X-ray powder diffraction pattern comprising peaks at 8.7±0.2° 2-θ, 21.6±0.2° 2-θ, and 24.4±0.2° 2-θ, as measured by X-ray powder diffraction using an X-ray wavelength of 1.5406 Å.

5. The crystalline N-(2,4,5-trifluoro-3-(3-morpholinoquinoxaline-6-carbonyl)phenyl)propane-1-sulfonamide of claim 4, wherein the X-ray powder diffraction pattern further comprises at least one peak selected from 17.5±0.2° 2-θ, 14.6±0.2° 2-θ, and 19.3±0.2° 2-θ, as measured by X-ray powder diffraction using an X-ray wavelength of 1.5406 Å.

6. The crystalline N-(2,4,5-trifluoro-3-(3-morpholinoquinoxaline-6-carbonyl)phenyl)propane-1-sulfonamide of claim 4 or claim 5, wherein the X-ray powder diffraction pattern further comprises at least one peak selected from 23.1±0.2° 2-θ, 16.0±0.2° 2-θ, and 25.7±0.2° 2-θ, as measured by X-ray powder diffraction using an X-ray wavelength of 1.5406 Å.

7. The crystalline N-(2,4,5-trifluoro-3-(3-morpholinoquinoxaline-6-carbonyl)phenyl)propane-1-sulfonamide of claim 4, wherein the X-ray powder diffraction pattern comprises at least five peaks selected from 8.7±0.2° 2-θ, 21.6±0.2° 2-θ, 24.4±0.2° 2-θ, 17.5±0.2° 2-θ, 14.6±0.2° 2-θ, 19.3±0.2° 2-θ, 23.1±0.2° 2-θ, 16.0±0.2° 2-θ, and 25.7±0.2° 2-θ, as measured by X-ray powder diffraction using an X-ray wavelength of 1.5406 Å.

8. The crystalline N-(2,4,5-trifluoro-3-(3-morpholinoquinoxaline-6-carbonyl)phenyl)propane-1-sulfonamide, of claim 4, wherein the X-ray powder diffraction pattern comprises peaks at 8.7±0.1° 2-θ, 21.6±0.1° 2-θ, 24.4±0.1° 2-θ, 17.5±0.1° 2-θ, 14.6±0.1° 2-θ, 19.3±0.1° 2-θ, 23.1±0.1° 2-θ, 16.0±0.1° 2-θ, and 25.7±0.1° 2-θ, as measured by X-ray powder diffraction using an X-ray wavelength of 1.5406 Å.

9. The crystalline N-(2,4,5-trifluoro-3-(3-morpholinoquinoxaline-6-carbonyl)phenyl)propane-1-sulfonamide of any one of claims 2 to 8, wherein Crystalline Form I is characterized by an X-ray powder diffraction pattern substantially the same as shown in FIG. 1.

10. The crystalline N-(2,4,5-trifluoro-3-(3-morpholinoquinoxaline-6-carbonyl)phenyl)propane-1-sulfonamide of any one of claims 2 to 9, wherein Crystalline Form I is characterized by a differential scanning calorimetry (DSC) thermogram comprising an endotherm in the range of about 180-190° C.

11. The crystalline N-(2,4,5-trifluoro-3-(3-morpholinoquinoxaline-6-carbonyl)phenyl)propane-1-sulfonamide of any one of claims 2 to 10, wherein Crystalline Form I is characterized by a differential scanning calorimetry (DSC) thermogram comprising an endotherm with an onset of about 184° C. and a peak of about 187° C.

12. The crystalline N-(2,4,5-trifluoro-3-(3-morpholinoquinoxaline-6-carbonyl)phenyl)propane-1-sulfonamide of any one of claims 2 to 11, wherein Crystalline Form I is characterized by a differential scanning calorimetry (DSC) thermogram substantially the same as shown in FIG. 2.

13. The crystalline N-(2,4,5-trifluoro-3-(3-morpholinoquinoxaline-6-carbonyl)phenyl)propane-1-sulfonamide of any one of claims 2 to 12, wherein Crystalline Form I is characterized by a Thermogravimetric Analysis (TGA) thermogram substantially the same as shown in FIG. 3.

14. The crystalline N-(2,4,5-trifluoro-3-(3-morpholinoquinoxaline-6-carbonyl)phenyl)propane-1-sulfonamide of claim 1, wherein the crystalline form is Crystalline Form II.

15. The crystalline N-(2,4,5-trifluoro-3-(3-morpholinoquinoxaline-6-carbonyl)phenyl)propane-1-sulfonamide of claim 14, wherein Crystalline Form II is characterized by:
(a) an X-ray powder diffraction pattern comprising peaks at 11.8±0.2° 2-θ, 8.6±0.2° 2-θ, and 15.1±0.2° 2-θ, as measured by X-ray powder diffraction using an X-ray wavelength of 1.5406 Å;
(b) an X-ray powder diffraction pattern substantially the same as shown in FIG. 4;
(c) a differential scanning calorimetry (DSC) thermogram comprising:
i) an endotherm in the range of about 135-145° C.;
ii) an exotherm in the range of about 143-153° C.; and
iii) an endotherm in the range of 155-165° C.;
(d) a differential scanning calorimetry (DSC) thermogram comprising
i) an endotherm with an onset of about 138° C. and a peak of about 143° C.;
ii) an exotherm with an onset of about 146° C. and a peak of about 148° C.; and
iii) an endotherm with an onset of about 158° C. and a peak of about 160° C.;
(e) a differential scanning calorimetry (DSC) thermogram substantially the same as shown in FIG. 5;
or
(f) combinations thereof.

16. The crystalline N-(2,4,5-trifluoro-3-(3-morpholinoquinoxaline-6-carbonyl)phenyl)propane-1-sulfonamide of claim 14 or claim 15, wherein Crystalline Form II is characterized by an X-ray powder diffraction pattern comprising peaks at 11.8±0.2° 2-θ, 8.6±0.2° 2-θ, and 15.1±0.2° 2-θ, as measured by X-ray powder diffraction using an X-ray wavelength of 1.5406 Å.

17. The crystalline N-(2,4,5-trifluoro-3-(3-morpholinoquinoxaline-6-carbonyl)phenyl)propane-1-sulfonamide of claim 16, wherein the X-ray powder diffraction pattern further comprises at least one peak selected from 17.2±0.2° 2-θ, 9.3±0.2° 2-θ, and 23.6±0.2° 2-θ, as measured by X-ray powder diffraction using an X-ray wavelength of 1.5406 Å.

18. The crystalline N-(2,4,5-trifluoro-3-(3-morpholinoquinoxaline-6-carbonyl)phenyl)propane-1-sulfonamide of claim 16 or claim 17, wherein the X-ray powder diffraction pattern further comprises at least one peak selected from 21.5±0.2° 2-θ, 22.2±0.2° 2-θ, and 14.3±0.2° 2-θ, as measured by X-ray powder diffraction using an X-ray wavelength of 1.5406 Å.

19. The crystalline N-(2,4,5-trifluoro-3-(3-morpholinoquinoxaline-6-carbonyl)phenyl)propane-1-sulfonamide of claim 16, wherein the X-ray powder diffraction pattern comprises at least five peaks selected from 11.8±0.2° 2-θ, 8.6±0.2° 2-θ, 15.1±0.2° 2-θ, 17.2±0.2° 2-θ, 9.3±0.2° 2-θ, 23.6±0.2° 2-θ, 21.5±0.2° 2-θ, 22.2±0.2° 2-θ, and 14.3±0.2° 2-θ, as measured by X-ray powder diffraction using an X-ray wavelength of 1.5406 Å.

20. The crystalline N-(2,4,5-trifluoro-3-(3-morpholinoquinoxaline-6-carbonyl)phenyl)propane-1-sulfonamide of claim 16, wherein the X-ray powder diffraction pattern comprises peaks at 11.8±0.1° 2-θ, 8.6±0.1° 2-θ, 15.1±0.1° 2-θ, 17.2±0.1° 2-θ, 9.3±0.1° 2-θ, 23.6±0.1° 2-θ, 21.5±0.1° 2-θ, 22.2±0.1° 2-θ, and 14.3±0.1° 2-θ, as measured by X-ray powder diffraction using an X-ray wavelength of 1.5406 Å.

21. The crystalline N-(2,4,5-trifluoro-3-(3-morpholinoquinoxaline-6-carbonyl)phenyl)propane-1-sulfonamide of any one of claims 14 to 20, wherein Crystalline Form II is characterized by an X-ray powder diffraction pattern substantially the same as shown in FIG. 4.

22. The crystalline N-(2,4,5-trifluoro-3-(3-morpholinoquinoxaline-6-carbonyl)phenyl)propane-1-sulfonamide of any one of claims 14 to 21, wherein Crystalline Form II is characterized by a differential scanning calorimetry (D S C) thermogram comprising:
i) an endotherm in the range of about 135-145° C.;
ii) an exotherm in the range of about 143-153° C.; and
iii) an endotherm in the range of 155-165° C.

23. The crystalline N-(2,4,5-trifluoro-3-(3-morpholinoquinoxaline-6-carbonyl)phenyl)propane-1-sulfonamide of any one of claims 14 to 22, wherein Crystalline Form II is characterized by a differential scanning calorimetry (D S C) thermogram comprising:
i) an endotherm with an onset of about 138° C. and a peak of about 143° C.;
ii) an exotherm with an onset of about 146° C. and a peak of about 148° C.; and
iii) an endotherm with an onset of about 158° C. and a peak of about 160° C.

24. The crystalline N-(2,4,5-trifluoro-3-(3-morpholinoquinoxaline-6-carbonyl)phenyl)propane-1-sulfonamide of any one of claims 14 to 23, wherein Crystalline Form II is characterized by a differential scanning calorimetry (DSC) thermogram substantially the same as shown in FIG. 5.

25. A pharmaceutical composition comprising crystalline N-(2,4,5-trifluoro-3-(3-morpholinoquinoxaline-6-carbonyl)phenyl)propane-1-sulfonamide of any one of claims 1 to 24, and at least one pharmaceutically acceptable excipient.

26. The pharmaceutical composition of claim 25, wherein the pharmaceutical composition is formulated for administration to a mammal by oral administration.

27. The pharmaceutical composition of claim 25 or claim 26, wherein the pharmaceutical composition is in the form of a solid form pharmaceutical composition.

28. The pharmaceutical composition of any one of claims 25 to 27, wherein the pharmaceutical composition is in the form of a tablet, a pill, or a capsule.

29. A packaged pharmaceutical composition comprising a pharmaceutical composition of any one of claim 25 or 28 and instructions for using the composition to treat a subject suffering from cancer.

30. A method of treating a neoplasm in a subject in need thereof, comprising administering to the subject a therapeutically effective amount of the crystalline N-(2,4,5-trifluoro-3-(3-morpholinoquinoxaline-6-carbonyl)phenyl)propane-1-sulfonamide of any one of claims 1 to 24, or the pharmaceutical composition of any one of claims 25 to 28.

31. The method of claim 30, wherein the neoplasm is a cancer.

32. The method of claim 31, wherein the cancer is colon carcinoma, pancreatic cancer, breast cancer, ovarian cancer, prostate cancer, thyroid cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, thyroid cancer, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung cancer, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, Langerhans cell histiocytosis (LCH), Erdheim-Chester disease (ECD), leukemia, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, or heavy chain disease.

33. The method of claim 31 or claim 32, wherein the cancer is melanoma.

34. The method of claim 33, wherein the melanoma is unresectable or metastatic melanoma.

35. The method of claim 31 or claim 32, wherein the cancer is non-small cell lung cancer.

36. The method of claim 31 or claim 32, wherein the cancer is colon cancer.

37. The method of claim 31 or claim 32, wherein the cancer is thyroid cancer.

38. The method of claim 31 or claim 32, wherein the cancer is ovarian cancer.

39. The method of claim 30, wherein the neoplasm is a benign tumor.

40. The method of claim 39, wherein the tumor is craniopharyngioma.

41. A method of preparing Crystalline Form I of N-(2,4,5-trifluoro-3-(3-morpholinoquinoxaline-6-carbonyl)phenyl)propane-1-sulfonamide, wherein the method comprises:
(a) dissolving the N-(2,4,5-trifluoro-3-(3-morpholinoquinoxaline-6-carbonyl)phenyl)propane-1-sulfonamide in a solvent to obtain a solution; and
(b) crystallizing the solution obtained in step (a) to obtain Crystalline Form I of N-(2,4,5-trifluoro-3-(3-morpholinoquinoxaline-6-carbonyl)phenyl)propane-1-sulfonamide.

42. The method of claim 41, wherein the solvent in step (a) comprises ethyl acetate, DCM, ethanol, or isopropanol.

43. The method of claim 41 or claim 42, wherein the solvent in step (a) comprises ethyl acetate.

44. The method of any one of claims 41 to 43, wherein step a is performed at a temperature of about 60-90° C.

45. The method of any one of claims 41 to 44, wherein step (a) is performed at a temperature of about 75-80° C.

46. The method of any one of claims 41 to 45, wherein step (a) is performed for a time of about 1-3 hours.

47. The method of any one of claims 41 to 46, wherein step (a) is performed for a time of about 2 hours.

48. The method of any one of claims 41 to 47, wherein step (b) comprises cooling the solution obtained in step (a) to room temperature.

49. The method of any one of claims 41 to 47, wherein step (b) comprises cooling the solution obtained in step (a) to a temperature of about 20-25° C.

50. The method of any one of claims 41 to 49, further comprising filtering the crystallized solution obtained in step (b) to obtain Crystalline Form I.

51. The method of any one of claims 41 to 50, further comprising drying the obtained Crystalline Form I.

52. The method of claim 51, wherein the drying is performed under vacuum at a temperature of about 40-50° C.

53. A method of preparing Crystalline Form II of N-(2,4,5-trifluoro-3-(3-morpholinoquinoxaline-6-carbonyl)phenyl)propane-1-sulfonamide, wherein the method comprises:
(a) dissolving the N-(2,4,5-trifluoro-3-(3-morpholinoquinoxaline-6-carbonyl)phenyl)propane-1-sulfonamide in a solvent to obtain a first solution;
(b) adding water to the first solution obtained in step (a) to form a mixture;
(c) separating the mixture obtained in step (b) into an organic phase and aqueous phase;
(d) isolating the solids from the organic phase;
(e) dissolving the solids obtained in step (d) in a second solvent to obtain a second solution; and
(f) crystallizing the second solution obtained in step (e) to obtain Crystalline Form II of N-(2,4,5-trifluoro-3-(3-morpholinoquinoxaline-6-carbonyl)phenyl)propane-1-sulfonamide.

54. The method of claim 53, wherein the solvent of step (a) comprises ethyl acetate, DCM, ethanol, or isopropanol.

55. The method of claim 53 or 54, wherein the amount of water added in step (b) is approximately 1:1 to 1:10 weight ratio of water:solvent added in step (a).

56. The method of any one of claims 53-55, wherein the amount of water added in step (b) is approximately 1:2 to 1:4 weight ratio of water:solvent added in step (a).

57. The method of any one of claims 53 to 56, wherein step (d) comprises concentrating the organic phase.

58. The method of claim 57, wherein the concentrating is performed under vacuum at a temperature of about 40-50° C.

59. The method of any one of claims 53 to 58, wherein the second solvent of step (e) comprises ethyl acetate, DCM, ethanol, or isopropanol.

60. The method of any one of claims 53 to 59, wherein the second solvent of step (e) is ethyl acetate.

61. The method of any one of claims 53 to 60, wherein step (f) comprises cooling the second solution obtained in step (e) to a temperature of about 10-20° C.

62. The method of claim 61, wherein the solution is maintained at a temperature of about 10-20° C. for about 3-5 hours.

63. The method of any one of claims 53 to 62, further comprising filtering the crystallized solution obtained in step (f) to obtain Crystalline Form II.

64. The method of any one of claims 53 to 63, further comprising drying the obtained Crystalline Form II.

65. The method of claim 64, wherein the drying is performed under vacuum at a temperature of about 50-60° C.
